(19) United States
(12) Patent Application Publication
(10) Pub. No.: US 2024/0374169 A1
(43) Pub. Date: Nov. 14, 2024

(54) CONTINUOUS MONITORING WITH NANO-DIAMOND HYDROGEL IN MICRONEEDLES

(71) Applicant: The University of Chicago, Chicago, IL (US)

(72) Inventors: Xiaoyang Wu, Chicago, IL (US); Jian Zhang, Chicago, IL (US)

(21) Appl. No.: 18/291,887

(22) PCT Filed: Jul. 29, 2022
(86) PCT No.: PCT/US2022/074292

§ 371 Date: Jan. 24, 2024

Related U.S. Application Data
(60) Provisional application No. 63/203,747, filed on Jul. 29, 2021.

(51) Int. Cl. A61B 5/145; A61B 5/1459

(52) U.S. Cl. CPC A61B 5/14514; A61B 5/14532; A61B 5/1459

(57) ABSTRACT

The present disclosure relates to a microneedle having a responsive hydrogel disposed therein. The responsive hydrogel can include optically active particles and capture agents. In particular, such a microneedle can be provided within a device or a monitoring system. Methods of using such microneedles are provided, such as for detection of an analyte.

### INCORPORATION BY REFERENCE

A PCT Request Form is filed concurrently with this specification as part of the present application. Each application that the present application claims benefit of or priority to as identified in the concurrently filed PCT Request Form is incorporated by reference herein in their entireties and for all purposes.

### STATEMENT OF GOVERNMENT INTEREST

This invention was made with government support under grant numbers R21AA077769, R01DA047785, and R01 OD023700 awarded by The National Institutes of Health. The government has certain rights in the invention.

### FIELD

The present disclosure relates to a microneedle having a hydrogel disposed therein. In particular, such a microneedle can be provided within a device or a monitoring system. Methods of using such microneedles are provided, such as for detection of an analyte.

### BACKGROUND

Point-of-care monitoring could enhance patient care. Continuous, non-invasive monitoring provides one avenue for such monitoring.

### SUMMARY

The present disclosure relates to a device useful for monitoring or detecting an analyte. In particular, the device employs microneedles to access interstitial fluid (ISF) from a subject in a minimally invasive manner. Generally, the microneedle is hollow and includes a responsive hydrogel disposed therein; and the hydrogel, in turn, includes optically active particles and capture agents. In use, analytes within the ISF are selectively captured by capture agents, thereby resulting in a physical change to the hydrogel that can be detected optically. Systems and methods employing such devices are also described herein.

In a first aspect, the present disclosure encompasses a device including: a microneedle including a wall and an internal bore surrounded by the wall; and a hydrogel (e.g., a responsive hydrogel) disposed within the internal bore. In some embodiments, the hydrogel includes a plurality of optically active particles and a plurality of capture agents.

In further embodiments, the device can include: an optical source configured to transmit an optical input signal to the microneedle; an optical sensor configured to receive an optical output signal from the microneedle; and a controller including a memory and a processor, wherein the controller is configured to be electrically connected to the optical source and the optical sensor. In some embodiments, the memory stores computer-executable instructions for controlling the processor to cause a sample within a microneedle to be analyzed by: transmitting the optical input signal from the optical source to the microneedle, thereby allowing the optical input signal to be transmitted through the hydrogel disposed within the microneedle; receiving the optical output signal from the microneedle to the optical sensor, wherein the optical output signal is indicative of a presence or an absence of an analyte captured within the hydrogel; obtaining data from the optical sensor indicating the presence or the absence of the analyte; and storing and/or transmitting the data.

In other embodiments, the device further includes: a wireless signal transmitter configured to transmit data from the controller or the memory to an external receiver.

In particular embodiments, the microneedle is configured to obtain a sample including interstitial fluid from a subject.

In a second aspect, the present disclosure encompasses a monitoring system including: (i) a sampling component (e.g., including a microneedle having a responsive hydrogel, such as any described herein) and (ii) a detecting component (e.g., configured to transmit and/or receive optical signals to and from the microneedle).

In some embodiments, the sample component includes: a microneedle including a wall and an internal bore surrounded by the wall; and a hydrogel disposed within the internal bore, wherein the hydrogel includes a plurality of optically active particles and a plurality of capture agents.

In some embodiments, the detecting component includes: an optical source configured to transmit an optical input signal to the microneedle; an optical sensor configured to receive an optical output signal from the microneedle; and a controller including a memory and a processor, wherein the controller is configured to be electrically connected to the optical source and the optical sensor.

In further embodiments, the monitoring system includes: a first optical fiber configured to provide optical communication between the optical source and the microneedle, wherein the first optical fiber is configured to transmit the optical input signal; and a second optical fiber configured to provide optical communication between the microneedle and the optical sensor, wherein the second optical fiber is configured to transmit the optical output signal.

In other embodiments, the monitoring system further includes: (iii) a communicating component, which can optionally include a wireless signal transmitter configured to transmit data from the controller or the memory to an external receiver.

In a third aspect, the present disclosure encompasses a method of detecting an analyte, the method including: applying a microneedle (e.g., any described herein) to a target site of a subject; and measuring one or more optical output signals transmitted from the microneedle, wherein the one or more optical output signals are indicative of a presence or an absence of the analyte captured within the hydrogel.

In particular embodiments, the target site is a dermal surface of the subject. In some embodiments, said applying includes providing access to interstitial fluid at the target site of the subject. In other embodiments, said applying includes affixing the microneedle at the target site for a period of about one to six months.

In some embodiments, said measuring includes detecting an optical emission intensity having a wavelength of about 450-485 nm.

In further embodiments, the method includes: analyzing the one or more optical output signals to determine the presence or the absence of the analyte, thereby providing processed data; and transmitting the processed data to an external receiver.

In any embodiment herein, the wall of the microneedle(s) includes an optically transparent material. In other embodiments, the wall includes a porous material. In yet other embodiments, the wall includes glass, sapphire, diamond, ruby, silica, polycarbonate, poly(dimethylsiloxane), poly(vinyl chloride), poly(methyl methacrylate), polyethylene, and combinations thereof.

In any embodiment herein, a plurality of microneedles is employed, wherein each of the plurality of microneedles includes an internal bore and a hydrogel disposed herein.

In any embodiment herein, the microneedle is configured to obtain a sample including interstitial fluid from a subject.

In any embodiment herein, the microneedle includes an anti-inflammatory coating, an anti-immunogenic coating, or a biocompatible coating.

In any embodiment herein, the microneedle extends from a planar substrate. In some embodiments, the planar substrate includes a flexible substrate (e.g., which in turn includes poly(dimethylsiloxane), poly(caprolactone), poly(lactic acid), or natural rubber (e.g., manufactured by casting, 3D printing, laser sintering, laser etching, and the like)).

In any embodiment herein, the wall includes an inner surface facing the internal bore, and wherein the inner surface is covalently bonded to the hydrogel. Non-limiting hydrogel can include, e.g., poly(acrylamide) (PAAm), poly(acrylic acid) (PAA), poly(ethylene oxide) (PEO), poly(ethylene oxide)-block-poly(acrylic acid) (PEO-b-PAA), poly(N-isopropylacrylamide) (PNIPAAm), poly(ethylene oxide)-block-poly(N-isopropylacrylamide) (PEO-b-pNIPAAm), poly[poly(ethylene glycol) diacrylate] (p[PEGDA]), poly(acrylamide-co-poly(ethylene glycol) diacrylate) (p[AAM-co-PEGDA]), poly(lactic acid) (PLA), poly(lactic-co-glycolic acid) (PLGA), poly(caprolactone) (PCL), poly(aniline) (PANI), poly(N-(3-amidino)-aniline), poly(octamethylene citric acid), alginate, a poloxamer, poly(dimethylsiloxane), poly(butadiene), poly(isoprene), or a copolymer thereof.

In any embodiment herein, the plurality of optically active particles includes an emission peak from about 450-485 nm. In other embodiments, the plurality of optically active particles includes a carbon-based material. In particular embodiments, the carbon-based material includes nanodiamonds, carbon nanotubes, carbon nanowires, or carbon particles.

In any embodiment herein, the plurality of capture agents is configured to bind to an analyte or detect a condition selected from the group consisting of an ion, a small molecule, a metal (e.g., a metal ion or a metal atom), a particle (e.g., a metal particle or a magnetic particle), a temperature, a peptide, a protein, a cytokine, a hydrophilic sample, or a hydrophobic sample. In some embodiments, the analyte is glucose, lactate, uric acid, glutathione, carbon dioxide, or hydrogen peroxide.

In any embodiment herein, the plurality of capture agents is selected from the group consisting of boronic acids, Schiff bases, acrylic acids, amides, amines, thiols, ionizable groups, reducible groups, charged groups, chelating groups, particles (e.g., magnetic particles), temperature responsive groups, redox indicators, photosensitizers, dyes, antibodies, nanostructures, and hydrophobic groups. Additional details are described herein.

### Definitions

As used herein, the term “about” is understood to account for minor increases and/or decreases beyond a recited value, which changes do not significantly impact the desired function of the parameter beyond the recited value(s). In some cases, “about” encompasses+/−10% of any recited value. As used herein, this term modifies any recited value, range of values, or endpoints of one or more ranges.

By “aliphatic” is meant a hydrocarbon group having at least one carbon atom to 50 carbon atoms (C₁₋₅₀), such as one to 25 carbon atoms (C₁₋₂₅), or one to ten carbon atoms (C₁₋₁₀), and which includes alkanes (or alkyl), alkenes (or alkenyl), alkynes (or alkynyl), including cyclic versions thereof, and further including straight- and branched-chain arrangements, and all stereo and position isomers as well. Such an aliphatic can be unsubstituted or substituted with one or more groups, such as groups described herein for an alkyl group. Aliphatic groups can include monovalent, bivalent, or multivalent forms.

By “alkoxy” is meant —OR, where R is an optionally substituted alkyl group, as described herein. Exemplary alkoxy groups include methoxy, ethoxy, butoxy, trihaloalkoxy, such as trifluoromethoxy, etc. The alkoxy group can be substituted or unsubstituted. For example, the alkoxy group can be substituted with one or more substitution groups, as described herein for alkyl. Exemplary unsubstituted alkoxy groups include C₁₋₃, C₁₋₆, C₁₋₁₂, C₁₋₁₆, C₁₋₁₈, C₁₋₂₀, or C₁₋₂₄ alkoxy groups.

By “alkenyl” is meant an optionally substituted C₂₋₂₄ alkyl group having one or more double bonds. The alkenyl group can be cyclic (e.g., C₃₋₂₄ cycloalkenyl) or acyclic. The alkenyl group can also be substituted or unsubstituted. For example, the alkenyl group can be substituted with one or more substitution groups, as described herein for alkyl. Non-limiting alkenyl groups include vinyl, allyl, and the like.

By “alkyl” and the prefix “alk” is meant a branched or unbranched saturated hydrocarbon group of 1 to 50 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic (e.g., C₃₋₅₀ cycloalkyl) or acyclic. The alkyl group can be branched or unbranched. The alkyl group can also be substituted or unsubstituted. For example, the alkyl group can be substituted with one, two, three or, in the case of alkyl groups of two carbons or more, four substituents independently selected from the group consisting of: (1) C₁₋₆ alkoxy (e.g., —O-Ak, wherein Ak is optionally substituted C₁₋₆ alkyl); (2) C₁₋₆ alkylsulfinyl (e.g., —S(O)-Ak, wherein Ak is optionally substituted C₁₋₆ alkyl); (3) C₁₋₆ alkylsulfonyl (e.g., —SO₂-Ak, wherein Ak is optionally substituted C₁₋₆ alkyl); (4) amino (e.g., —NR^{N1}R^{N2}, where each of R^{N1} and R^{N2} is, independently, H or optionally substituted alkyl, or R^{N1} and R^{N2}, taken together with the nitrogen atom to which each are attached, form a heterocyclyl group); (5) aryl; (6) arylalkoxy (e.g., —O-L-Ar, wherein L is a bivalent form of optionally substituted alkyl and Ar is optionally substituted aryl); (7) aryloyl (e.g., —C(O)—Ar, wherein Ar is optionally substituted aryl); (8) azido (e.g., —N₃); (9) cyano (e.g., —CN); (10) carboxyaldehyde (e.g., —C(O)H); (11) C₃₋₈ cycloalkyl (e.g., a monovalent saturated or unsaturated non-aromatic cyclic C₃₋₈ hydrocarbon group); (12) halo (e.g., F, Cl, Br, or I); (13) heterocyclyl (e.g., a 5-, 6- or 7-membered ring, unless otherwise specified, containing one, two, three, or four non-carbon heteroatoms, such as nitrogen, oxygen, phosphorous, sulfur, or halo); (14) heterocyclyloxy (e.g., —O-Het, wherein Het is heterocyclyl, as described herein); (15) heterocyclyloyl (e.g., —C(O)—Het, wherein Het is heterocyclyl, as described herein); (16) hydroxyl (e.g., —OH); (17) N-protected amino; (18) nitro (e.g., —NO₂); (19) oxo (e.g., ═O); (20) C₃₋₈ spirocyclyl (e.g., an alkylene or heteroalkylene diradical, both ends of which are bonded to the same carbon atom of the parent group); (21) C₁₋₆ thioalkoxy (e.g., —S-Ak, wherein Ak is optionally substituted C₁₋₆ alkyl); (22) thiol (e.g., —SH); (23) —CO₂R^{A}, where R^{A} is selected from the group consisting of (a) hydrogen, (b) C₁₋₆ alkyl, (c) C₄₋₁₈ aryl, and (d) (C₄₋₁₈ aryl) C₁₋₆ alkyl (e.g., -L-Ar, wherein L is a bivalent form of optionally substituted alkyl group and Ar is optionally substituted aryl); (24) —C(O)NR^{B}R^{C}, where each of RB and R^{c} is, independently, selected from the group consisting of (a) hydrogen, (b) C₁₋₆ alkyl, (c) C₄₋₁₈ aryl, and (d) (C₄₋₁₈ aryl) C₁₋₆ alkyl (e.g., -L-Ar, wherein L is a bivalent form of optionally substituted alkyl group and Ar is optionally substituted aryl); (25) —SO₂RD, where RD is selected from the group consisting of (a) C₁₋₆ alkyl, (b) C₄₋₁₈ aryl, and (c) (C₄₋₁₈ aryl) C₁₋₆ alkyl (e.g., -L-Ar, wherein L is a bivalent form of optionally substituted alkyl group and Ar is optionally substituted aryl); (26) —SO₂NR^{E}R^{F}, where each of R^{E} and R^{F} is, independently, selected from the group consisting of (a) hydrogen, (b) C₁₋₆ alkyl, (c) C₄₋₁₈ aryl, and (d) (C₄₋₁₈ aryl) C₁₋₆ alkyl (e.g., -L-Ar, wherein L is a bivalent form of optionally substituted alkyl group and Ar is optionally substituted aryl); and (27) —NR^{G}R^{H}, where each of R^{G} and R^{H} is, independently, selected from the group consisting of (a) hydrogen, (b) an N-protecting group, (c) C₁₋₆ alkyl, (d) C₂₋₆ alkenyl (e.g., optionally substituted alkyl having one or more double bonds), (e) C₂₋₆ alkynyl (e.g., optionally substituted alkyl having one or more triple bonds), (f) C₄₋₁₈ aryl, (g) (C₄₋₁₈ aryl) C₁₋₆ alkyl (e.g., L-Ar, wherein L is a bivalent form of optionally substituted alkyl group and Ar is optionally substituted aryl), (h) C₃₋₈ cycloalkyl, and (i) (C₃₋₈ cycloalkyl) C₁₋₆ alkyl (e.g., -L-Cy, wherein L is a bivalent form of optionally substituted alkyl group and Cy is optionally substituted cycloalkyl, as described herein), wherein in one embodiment no two groups are bound to the nitrogen atom through a carbonyl group or a sulfonyl group. The alkyl group can be a primary, secondary, or tertiary alkyl group substituted with one or more substituents (e.g., one or more halo or alkoxy). In some embodiments, the unsubstituted alkyl group is a C₁₋₃, C₁₋₆, C₁₋₁₂, C₁₋₁₆, C₁₋₁₈, C₁₋₂₀, C₁₋₂₄, C₁₋₃₂, C₁₋₃₈, C₁₋₄₂, C₁₋₅₀, C₂₋₃, C₂₋₆, C₂₋₁₂, C₂₋₁₆, C₂₋₁₈, C₂₋₂₀, C₂₋₂₄, C₂₋₃₂, C₂₋₃₈, C₂₋₄₂, C₂₋₅₀, C₈₋₅₀, C₁₀₋₅₀, C₁₅₋₅₀, C₂₀₋₅₀, C₂₅₋₅₀, C₃₀₋₅₀, or C₃₅₋₅₀ alkyl group.

By “alkylene” is meant a multivalent (e.g., bivalent) form of an alkyl group, as described herein. Non-limiting alkylene groups include methylene, ethylene, propylene, butylene, etc. In some embodiments, the alkylene group is a C₁₋₃, C₁₋₆, C₁₋₁₂, C₁₋₁₆, C₁₋₁₈, C₁₋₂₀, C₁₋₂₄, C₁₋₃₂, C₁₋₃₈, C₁₋₄₂, C₁₋₅₀, C₂₋₃, C₂₋₆, C₂₋₁₂, C₂₋₁₆, C₂₋₁₈, C₂₋₂₀, C₂₋₂₄, C₂₋₃₂, C₂₋₃₈, C₂₋₄₂, C₂₋₅₀, C₁₀₋₅₀, C₁₅₋₅₀, C₂₀₋₅₀, C₂₅₋₅₀, C₃₀₋₅₀, or C₃₅₋₅₀ alkylene group. The alkylene group can be branched or unbranched. The alkylene group can also be substituted or unsubstituted. For example, the alkylene group can be substituted with one or more substitution groups, as described herein for alkyl.

By “alkyleneoxy” is meant an alkylene group, as defined herein, attached to the parent molecular group through an oxygen atom.

By “alkynyl” is meant an optionally substituted C₂₋₂₄ alkyl group having one or more triple bonds. The alkynyl group can be cyclic or acyclic and is exemplified by ethynyl, 1-propynyl, and the like. The alkynyl group can also be substituted or unsubstituted. For example, the alkynyl group can be substituted with one or more substitution groups, as described herein for alkyl.

By “amino” is meant —NR^{N1}R^{N2}, where each of R^{N1} and R^{N2} is, independently, H, optionally substituted alkyl, or optionally substituted aryl, or R^{N1} and R^{N2}, taken together with the nitrogen atom to which each are attached, form a heterocyclyl group, as defined herein.

By “aromatic” is meant a cyclic, conjugated group or moiety of, unless specified otherwise, from 5 to 15 ring atoms having a single ring (e.g., phenyl) or multiple condensed rings in which at least one ring is aromatic (e.g., naphthyl, indolyl, or pyrazolopyridinyl); that is, at least one ring, and optionally multiple condensed rings, have a continuous, delocalized π-electron system. Typically, the number of out of plane π-electrons corresponds to the Huckel rule (4π+2). The point of attachment to the parent structure typically is through an aromatic portion of the condensed ring system. Such an aromatic can be unsubstituted or substituted with one or more groups, such as groups described herein for an alkyl group. Yet other substitution groups can include aliphatic, haloaliphatic, halo, nitrate, cyano, sulfonate, sulfonyl, or others. Non-limiting aromatic can include aryl and heteroaryl groups. Aromatic groups can include monovalent, bivalent, or multivalent forms.

By “aryl” is meant a group that contains any carbon-based aromatic group including, but not limited to, phenyl, benzyl, anthracenyl, anthryl, benzocyclobutenyl, benzocyclooctenyl, biphenylyl, chrysenyl, dihydroindenyl, fluoranthenyl, indacenyl, indenyl, naphthyl, phenanthryl, phenoxybenzyl, picenyl, pyrenyl, terphenyl, and the like, including fused benzo-C₄₋₈ cycloalkyl radicals (e.g., as defined herein) such as, for instance, indanyl, tetrahydronaphthyl, fluorenyl, and the like. The term aryl also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term non-heteroaryl, which is also included in the term aryl, defines a group that contains an aromatic group that does not contain a heteroatom. The aryl group can be substituted or unsubstituted. The aryl group can be substituted with one, two, three, four, or five substituents provided herein as possible substitutions for alkyl. In particular embodiments, an unsubstituted aryl group is a C₄₋₁₈, C₄₋₁₄, C₄₋₁₂, C₄₋₁₀, C₆₋₁₈, C₆₋₁₄, C₆₋₁₂, or C₆₋₁₀ aryl group.

By “arylene” is meant a multivalent (e.g., bivalent form) of an aryl group, as defined herein. The arylene group can be substituted or unsubstituted. For example, the arylene group can be substituted with one or more substitution groups, as described herein for alkyl.

By “aryloxy” is meant —OR, where R is an optionally substituted aryl group, as described herein. In some embodiments, an unsubstituted aryloxy group is a C₄₋₁₈ or C₆₋₁₈ aryloxy group.

By “azido” is meant an —N₃ group.

By “carboxyl” is meant a —CO₂H group.

By “cyano” is meant a —CN group.

By “ester” as used herein is represented by the formula —OC(O)A₁ or —C(O)OA₁, where A₁ can be an optionally substituted aliphatic, as described herein. In some non-limiting embodiments, A₁ is optionally substituted alkyl.

By “halo” is meant F, Cl, Br, or I.

By “heteroaliphatic” is meant an aliphatic group, as defined herein, containing one, two, three, or four non-carbon heteroatoms (e.g., independently selected from the group consisting of nitrogen, oxygen, phosphorous, sulfur, selenium, or halo). The heteroaliphatic group can be substituted or unsubstituted. For example, the heteroaliphatic group can be substituted with one or more substitution groups, as described herein for alkyl. Heteroaliphatic groups can include monovalent, bivalent, or multivalent forms.

By “heteroalkylene” is meant a multivalent (e.g., bivalent form) of an alkyl group, as defined herein, containing one, two, three, or four non-carbon heteroatoms (e.g., independently selected from the group consisting of nitrogen, oxygen, phosphorous, sulfur, selenium, or halo). The heteroalkylene group can be substituted or unsubstituted. For example, the heteroalkylene group can be substituted with one or more substitution groups, as described herein for alkyl.

By “heterocyclyl” is meant a 3-, 4-, 5-, 6- or 7-membered ring (e.g., a 5-, 6- or 7-membered ring), unless otherwise specified, containing one, two, three, or four non-carbon heteroatoms (e.g., independently selected from the group consisting of nitrogen, oxygen, phosphorous, sulfur, selenium, or halo). The 3-membered ring has zero to one double bonds, the 4- and 5-membered ring has zero to two double bonds, and the 6- and 7-membered rings have zero to three double bonds. The term “heterocyclyl” also includes bicyclic, tricyclic and tetracyclic groups in which any of the above heterocyclic rings is fused to one, two, or three rings independently selected from the group consisting of an aryl ring, a cyclohexane ring, a cyclohexene ring, a cyclopentane ring, a cyclopentene ring, and another monocyclic heterocyclic ring, such as indolyl, quinolyl, isoquinolyl, tetrahydroquinolyl, benzofuryl, benzothienyl and the like.

By “hydroxyl” is meant —OH.

By “hydroxyalkyl” is meant an alkyl group, as defined herein, substituted by one to three hydroxyl groups, with the proviso that no more than one hydroxyl group may be attached to a single carbon atom of the alkyl group and is exemplified by hydroxymethyl, dihydroxypropyl, and the like.

By “leaving group” is meant an atom (or a group of atoms) with electron withdrawing ability that can be displaced as a stable species, taking with it the bonding electrons. Examples of suitable leaving groups include halides and sulfonates including, but not limited to, triflate (—OTf), mesylate (—OMs), tosylate (—OTs), brosylate (—OBs), Cl, Br, and I.

By “nitro” is meant an —NO₂ group.

By “salt” is meant an ionic form of a compound or structure (e.g., any formulas, compounds, or compositions described herein), which includes a cation or anion compound to form an electrically neutral compound or structure. Salts are well known in the art. For example, non-toxic salts are described in Berge S M et al., “Pharmaceutical salts,” J. Pharm. Sci. 1977 January; 66(1):1-19; and in “Handbook of Pharmaceutical Salts: Properties, Selection, and Use,” Wiley-VCH, April 2011 (2nd rev. ed., eds. P. H. Stahl and C. G. Wermuth. The salts can be prepared in situ during the final isolation and purification of the compounds of the invention or separately by reacting the free base group with a suitable organic acid (thereby producing an anionic salt) or by reacting the acid group with a suitable metal or organic salt (thereby producing a cationic salt). Representative anionic salts include acetate, adipate, alginate, ascorbate, aspartate, benzenesulfonate, benzoate, bicarbonate, bisulfate, bitartrate, borate, bromide, butyrate, camphorate, camphorsulfonate, chloride, citrate, cyclopentanepropionate, digluconate, dihydrochloride, diphosphate, dodecylsulfate, edetate, ethanesulfonate, fumarate, glucoheptonate, gluconate, glutamate, glycerophosphate, hemisulfate, heptonate, hexanoate, hydrobromide, hydrochloride, hydroiodide, hydroxyethanesulfonate, hydroxynaphthoate, iodide, lactate, lactobionate, laurate, lauryl sulfate, malate, maleate, malonate, mandelate, mesylate, methanesulfonate, methylbromide, methylnitrate, methylsulfate, mucate, 2-naphthalenesulfonate, nicotinate, nitrate, oleate, oxalate, palmitate, pamoate, pectinate, persulfate, 3-phenylpropionate, phosphate, picrate, pivalate, polygalacturonate, propionate, salicylate, stearate, subacetate, succinate, sulfate, tannate, tartrate, theophyllinate, thiocyanate, triethiodide, toluenesulfonate, undecanoate, valerate salts, and the like. Representative cationic salts include metal salts, such as alkali or alkaline earth salts, e.g., barium, calcium (e.g., calcium edetate), lithium, magnesium, potassium, sodium, and the like; other metal salts, such as aluminum, bismuth, iron, and zinc; as well as nontoxic ammonium, quaternary ammonium, and amine cations, including, but not limited to ammonium, tetramethylammonium, tetraethylammonium, methylamine, dimethylamine, trimethylamine, triethylamine, ethylamine, pyridinium, and the like. Other cationic salts include organic salts, such as chloroprocaine, choline, dibenzylethylenediamine, diethanolamine, ethylenediamine, methylglucamine, and procaine. Yet other salts include ammonium, sulfonium, sulfoxonium, phosphonium, iminium, imidazolium, benzimidazolium, amidinium, guanidinium, phosphazinium, phosphazenium, pyridinium, etc., as well as other cationic groups described herein (e.g., optionally substituted isoxazolium, optionally substituted oxazolium, optionally substituted thiazolium, optionally substituted pyrrolium, optionally substituted furanium, optionally substituted thiophenium, optionally substituted imidazolium, optionally substituted pyrazolium, optionally substituted isothiazolium, optionally substituted triazolium, optionally substituted tetrazolium, optionally substituted furazanium, optionally substituted pyridinium, optionally substituted pyrimidinium, optionally substituted pyrazinium, optionally substituted triazinium, optionally substituted tetrazinium, optionally substituted pyridazinium, optionally substituted oxazinium, optionally substituted pyrrolidinium, optionally substituted pyrazolidinium, optionally substituted imidazolinium, optionally substituted isoxazolidinium, optionally substituted oxazolidinium, optionally substituted piperazinium, optionally substituted piperidinium, optionally substituted morpholinium, optionally substituted azepanium, optionally substituted azepinium, optionally substituted indolium, optionally substituted isoindolium, optionally substituted indolizinium, optionally substituted indazolium, optionally substituted benzimidazolium, optionally substituted isoquinolinum, optionally substituted quinolizinium, optionally substituted dehydroquinolizinium, optionally substituted quinolinium, optionally substituted isoindolinium, optionally substituted benzimidazolinium, and optionally substituted purinium).

By “sulfo” or “sulfonic acid” is meant an —S(O)₂OH group.

By “thiol” is meant an —SH group.

By “attaching,” “attachment,” “linked,” “linking,” or related word forms is meant any covalent or non-covalent bonding interaction between two components. Non-covalent bonding interactions include, without limitation, hydrogen bonding, ionic interactions, halogen bonding, electrostatic interactions, π bond interactions, hydrophobic interactions, inclusion complexes, clathration, van der Waals interactions, and combinations thereof.

As used herein, the terms “top,” “bottom,” “upper,” “lower,” “above,” and “below” are used to provide a relative relationship between structures. The use of these terms does not indicate or require that a particular structure must be located at a particular location in the apparatus.

By “optical communication,” as used herein, refers to any optical element, optical component, and/or pathway (e.g., in air or a vacuum) through which an optical signal (e.g., any illumination with electromagnetic radiation, e.g., ultraviolet, visible, near-infrared, etc.) may pass substantially unrestricted when the pathway is open.

By “fluidic communication,” as used herein, refers to any duct, channel, tube, pipe, reservoir, chamber, or pathway through which a substance, such as a liquid, gas, or solid may pass substantially unrestricted when the pathway is open. When the pathway is closed, the substance is substantially restricted from passing through. Typically, limited diffusion of a substance through the material of a plate, base, and/or a substrate, which may or may not occur depending on the compositions of the substance and materials, does not constitute fluidic communication.

By “micro” is meant having at least one dimension that is less than 1 mm. For instance, a microstructure (e.g., any structure described herein) can have a length, width, height, cross-sectional dimension, circumference, radius (e.g., external or internal radius), or diameter that is less than 1 mm. In another instance, a microneedle can have a length, width, height, cross-sectional dimension, circumference, radius (e.g., external or internal radius), and/or diameter that is less than 1 mm.

By “nano” is meant having at least one dimension that is less than 1 μm. For instance, a nanostructure (e.g., any structure described herein) can have a length, width, height, cross-sectional dimension, circumference, radius (e.g., external or internal radius), or diameter that is less than 1 μm.

By “treating” a disease, disorder, or condition in a subject is meant reducing at least one symptom of the disease, disorder, or condition by administrating a therapeutic substance to the subject.

By “treating prophylactically” a disease, disorder, or condition in a subject is meant reducing the frequency of occurrence or severity of (e.g., preventing) a disease, disorder or condition by administering to the subject a therapeutic substance to the subject prior to the appearance of a disease symptom or symptoms.

By “sample” is meant any specimen obtained from a subject, a plant, an environment, a chemical material, a biological material, or a manufactured product. The sample can include any useful material, such as biological (e.g., genomic) and/or chemical matter.

By “subject” is meant a human or non-human animal (e.g., a mammal). Exemplary non-human animals include livestock (e.g., cattle, goat, sheep, pig, poultry, farm fish, etc.), domestic animals (e.g., dog, cat, etc.), or captive wild animals (e.g., a zoo animal).

These and other aspects are described further below with reference to the drawings.

### BRIEF DESCRIPTION OF THE DRAWINGS

FIG. 1A-1C shows schematics of non-limiting devices and microneedles. Provided are (A) a non-limiting device including a microneedle 124; (B) a non-limiting design of a device including a light source and an optical sensor on a mini size chip with a wireless signal transmitter; and (C) a non-limiting microneedle having a wall 144 and a responsive hydrogel 145 disposed within an internal bore surrounded by the wall 144.

FIG. 2A-2C shows schematics of a non-limiting particles and responsive hydrogels. Provided are (A) a non-limiting particle 201; (B) a non-limiting responsive hydrogel in a hydrogel with increasing or decreasing stimuli; and (C) a non-limiting responsive hydrogel including a hydrogel 234a/b, a capture agent 236a/b, an optically active particle 231a/b, and an analyte 238 that binds the capture agent 236a.

FIG. 3A-3B shows schematics of a non-limiting particles and responsive hydrogels. Provided are (A) a non-limiting particle that is a functionalize nanodiamond 301; and (B) a non-limiting stimuli-responsive nanodiamond hydrogel 350.

FIG. 4A-4B shows non-limiting schematics of (A) binding between glucose and boronic acid, an example of a capture agent; and (B) responsive fluorescent changes of the nanodiamond hydrogel in response to glucose.

FIG. 5 shows a photograph of a prototype microneedle.

FIG. 6 shows a non-limiting schematic of the synthesis of blue fluorescent diamond.

FIG. 7 shows a non-limiting schematic of the preparation of responsive nanodiamond hydrogel in a glass microneedle

FIG. 8A-8B shows the optical response of in vitro use of a microneedle having a glucose-responsive hydrogel. Provided are (A) corresponding emission intensity as a function of changing glucose concentration; and (B) reversible fluorescent changes upon exposure to a glucose solution (200 mg/dL) or a buffer solution (PBS).

FIG. 9 shows fluorescent intensity traces of blood glucose concentration in an in vivo experiment.

FIG. 10A-10D shows fluorescent intensity traces of blood glucose concentration in in vivo experiments on glucose-challenged or insulin-challenged mice.

FIG. 11A shows fluorescent intensity traces of blood glucose concentration in in vivo experiments on a glucose-challenged on day zero.

FIG. 11B shows fluorescent intensity traces of blood glucose concentration in in vivo experiments on a glucose-challenged on day three.

FIG. 11C shows representative hematoxylin and eosin staining of porcine skin at day zero (E), at day seven (F), three days after microneedle removal (G) and ten days after microneedle removal.

FIG. 12A shows an electron microscopic image of a fluorescent nano-diamond.

FIG. 12B shows a graph of the dynamic light scattering data for a fluorescent nano-diamond.

FIG. 12C shows a graph of the emission spectral data for a fluorescent nano-diamond at 400 nm excitation.

FIG. 12D shows a graph of the emission spectral data for of a fluorescent nano-diamond at 370 nm excitation (slope on the right) and at 450 nm excitation (slope on the left).

### DETAILED DESCRIPTION

In the following description, numerous specific details are set forth to provide a thorough understanding of the presented embodiments. The disclosed embodiments may be practiced without some or all of these specific details. In other instances, well-known process operations have not been described in detail to not unnecessarily obscure the disclosed embodiments. While the disclosed embodiments will be described in conjunction with the specific embodiments, it will be understood that it is not intended to limit the disclosed embodiments.

The present disclosure relates to a microneedle having a responsive hydrogel disposed therein. In particular embodiments, the responsive hydrogel includes a polymer-based backbone to provide a hydrogel structure, a plurality of capture agents configured to bind to a desired analyte, and a plurality of optically active particles configured to provide an optically detectable signal in response to the presence of the analyte. Details regarding the components for responsive hydrogels are further described herein.

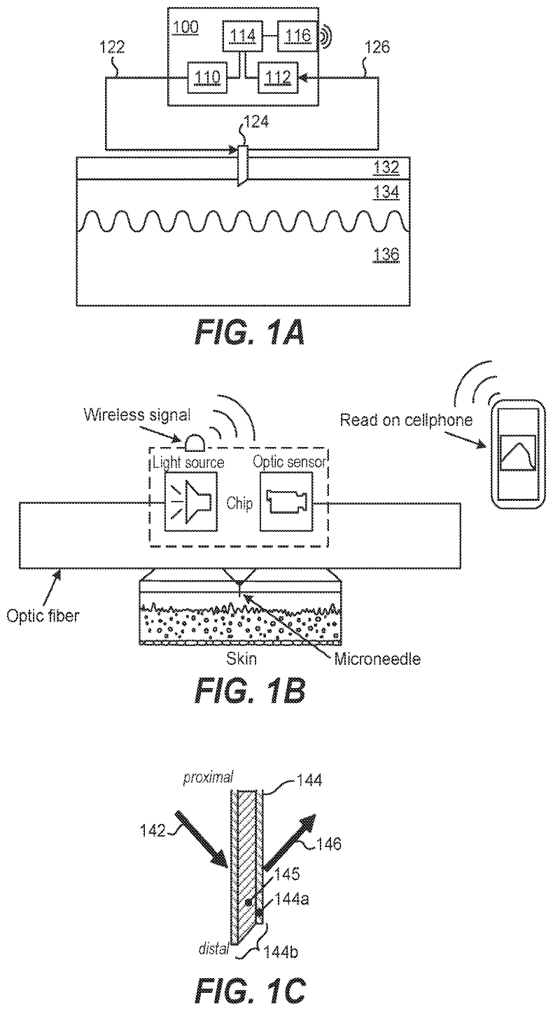

The microneedle can be configured to access interstitial fluid (ISF) from a subject. In some non-limiting instances, to minimize pain, the microneedle is configured to not interact with deeper layers of the dermis. For instance, as seen in FIG. 1A, the skin can be approximated as having various layers, including the epidermis 132, 134 (e.g., having a thickness of 0.05 to 1.5 mm, in which the stratum corneum 132 has a thickness of about 10 and 40 μm) and the dermis 136 (e.g., having a thickness of 0.3 to 3 mm). Accordingly, to obtain fluid in proximity to the basement membrane of the epidermis layer, the needle can be optimized to have a length that is more than about 0.2 mm, 0.3 mm, 0.5 mm, 1 mm, or 1.5 mm, depending on the desired location of the device on the body. Furthermore, to obtain fluid in the dermis layer, the needle can be optimized to have a length that is more than about 0.3 mm, 0.5 mm, 1 mm, 1.5 mm, 2 mm, 2.5 mm, or 3 mm, depending on the desired location of the device on the body. A desired cross-sectional dimension can be determined by the skin site to be sampled (e.g., a dimension to allow for local testing of the subject, while minimizing pain), by the desired flow rate of the sample within the internal bore of the needle (e.g., the flow rate can be optimized to allow for obtaining a fluid within a particular sampling time, or to minimize sample contamination, coagulation, and/or discomfort to the subject), by the desired volume of sample to be collected, etc.

The microneedle can be integrated in any useful device. FIG. 1A provides a non-limiting example of a device including a microneedle 124, an optical source 110 configured to transmit an optical input signal to the microneedle 124, and an optical sensor 112 configured to receive an optical output signal from the microneedle 124. Such optical signals (e.g., optical input or output signals) can be transmitted or received by any useful optical relay component 122, 126, such as optical fibers, optical waveguides, mirrors, and the like that can provide optical communication.

The microneedle can be associated with a housing 100 that includes components for use with the microneedle 124. For instance, the housing 100 can include the optical source 110, a first port configured to connect a first optical relay component 122 from the optical source 110 to the microneedle 124, the optical sensor 112 (or optical detector), a second port configured to connect to a second optical relay component 126 from the microneedle 124 to the optical sensor 112, a controller 114, and a wireless signal transmitter 116 configured to transmit data from the controller 114 (or a component thereof, such as a memory) to an external receiver. The housing can be provided to protect a substrate or a chip, which in turn includes integrated hardware components, such as the controller(s), optical source(s), optical sensor(s), and optical relay components. The housing (and components therein) can be configured for long-term detection of the target analyte. Further details on hardware components are provided herein. FIG. 1B provides another schematic of a non-limiting device.

In use, the microneedle includes a responsive hydrogel that responds to the presence of a target analyte. FIG. 1C shows a non-limiting microneedle 144 having a wall 144a, an internal bore surrounded by the wall 144a, and a puncturing edge 144b disposed at the distal end of the microneedle 144. Disposed within the internal bore is a responsive hydrogel 145. In use, an optical input signal 142 is transmitted to the microneedle 144, through the microneedle wall 144a, and then through the responsive hydrogel 145. Then, an optical output signal 146 is emitted from the microneedle 144.

The puncturing edge 144b is configured to be applied to skin and to extract ISF from the subject. The ISF can then enter the internal bore of the microneedle and interact with the responsive hydrogel. If the target analyte is present in the ISF, then one or more physical properties of the hydrogel is modified, such that the optical output signal 146 differs from the optical input signal 142. Such physical properties can include changes in turbidity, refractive index, solubility, and the like, which can be detected by measuring optical density, optical intensity, etc.

Various components within the responsive hydrogel provide such a response. For example, the polymer-based backbone of the hydrogel can provide a three-dimensional network, upon which functional groups can be added. Such functional groups can include side-chains (e.g., hydrophobic side chains) that affect the extent of heterogeneities within the network. This, in turn, can affect the turbidity (or other optical characteristics) of the nascent polymer network (prior to exposure to the target analyte).

The polymer network can also employ other functional groups, such as polymerizable or linker groups that allow for covalent attachment of the polymer to the internal wall of the microneedle. For instance, the inner surface of the internal bore (within the hollow microneedle) can be surface modified to allow for cross-linking with the polymerizable groups or linker groups that are pendant from the backbone of the hydrogel.

Yet other functional groups can include capture agents that are linked to the polymer network. Such capture agents can bind to target analytes, if present. Such binding or capture events can affect the side-chain interactions within the polymer network, which in turn can affect its optical characteristics. For example, the nascent polymer can be a turbid polymer due to hydrophobic interactions between the side-chains within the polymer network that provide local microheterogeneities. Upon binding the target analyte, such hydrophobic interactions can be disrupted, so that the polymer network is more optically clear. Other interactions (e.g., hydrophobic, hydrophilic, anionic, cationic, etc.) can be used in combination to transition the polymer network between disordered and ordered configurations or between collapsed and expanded configurations that are elicited upon binding to a target analyte.

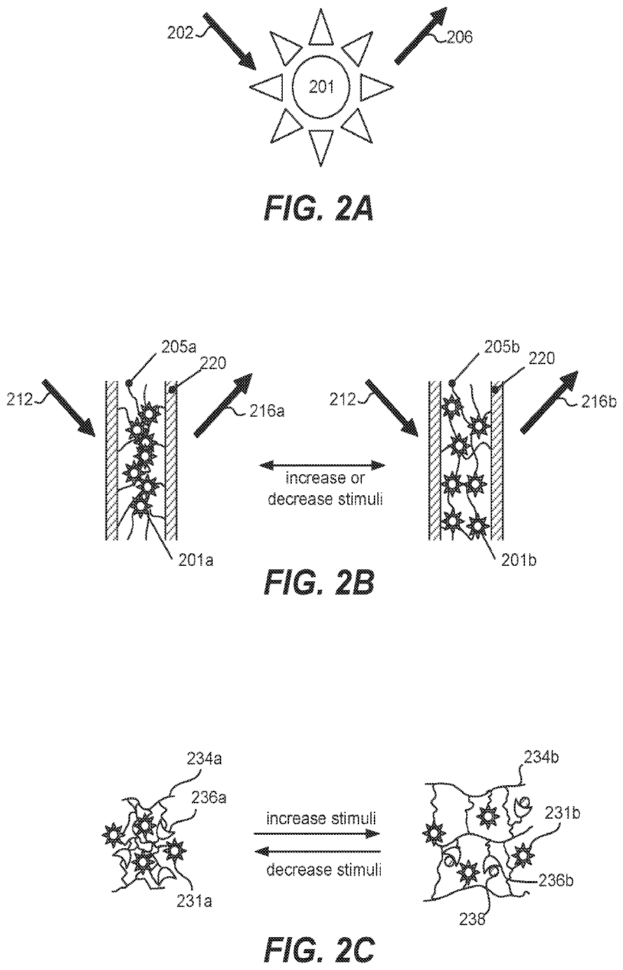

The responsive hydrogel further includes optically active particles entrapped therein. Whereas the capture agents confer specificity to a particular target analyte, the particles provide a detectable signal. For instance, if binding to a target analyte provides a more optically clear hydrogel, then optical emissions from the entrapped particles can be more readily observed or detected. In this way, an increased presence of the analyte will provide an increased optical intensity from the particles. Such particles can have any useful shape (e.g., sphere, tube, stellate, etc.) and configuration (e.g., quantum dot, colloidal particle, fluorescent particle, luminescent particle, chemiluminescent particle, and the like). FIG. 2A provides a schematic of a non-limiting optically active particle 201 that can be exposed to an optical input signal 202 to provide a resulting optical output signal 206.

FIG. 2B provides a schematic of a non-limiting responsive hydrogel that reacts to an increase or decrease in stimuli. Such stimuli can include exposure to a target analyte (e.g., glucose) or exposure to a condition (e.g., temperature). As can be seen, optically active particles 201a are entrapped in the responsive hydrogel 205a, which is attached to the inner wall 220 of the microneedle. Exposing the microneedle to an optical input signal 212 provides a first optical output signal 216a. Upon increasing or decreasing stimuli, the responsive hydrogel provides a detectable optical response. For instance, the responsive hydrogel can possess a change in refractive index or an attenuation in light transmission. As can be seen, after the change in stimuli, the responsive hydrogel 205b provides an increased optical transmission of emission from the particles 205b, such that the second optical output signal 216b is different than the first optical output signal 216a.

In one instance, as seen in FIG. 2C, an increase in stimuli is the increased presence of the target analyte 238. In the absence of target analyte, the nascent responsive hydrogel 234a includes unbound capture agents 236a and particles 231a entrapped in an optically turbid polymer network. In the presence of target analyte 238, the responsive hydrogel 234b includes bound capture agents 236b and particles 231b entrapped in an optically clear polymer network. In this way, an increase of the target analyte can be correlated with an increase in optical output signal emitted by the particles.

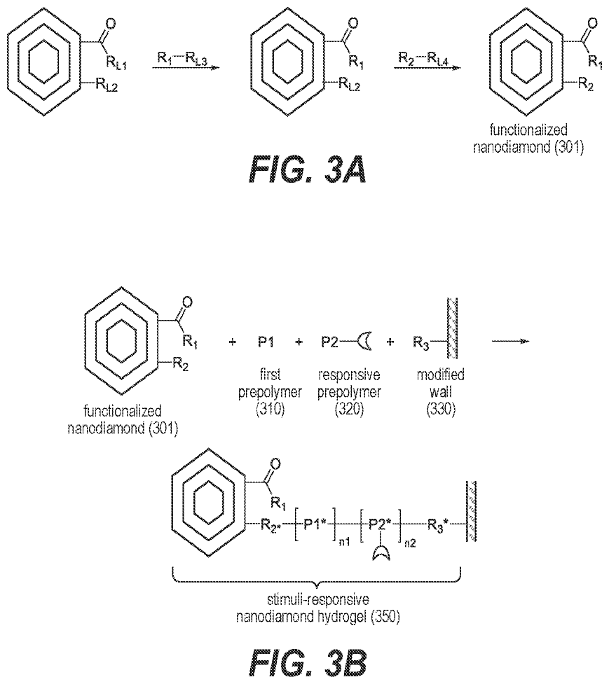

FIG. 3A-3B shows a non-limiting method of preparing an optically active particle, as well as a responsive hydrogel including such particles. As seen in FIG. 3A, the particle can be a nanodiamond, which can be treated to provide a functionalized nanodiamond 301. For instance, the nanodiamond can have one or more surface-accessible groups (e.g., —C(O)—R_{L1} and/or —R_{L2}, in which each of L1 and L2 is, independently, a leaving group or a reactive group, such as any described herein). Such surface-accessible groups can be further reacted to provide further functional groups (e.g., any described herein). For example, further functional groups can include those to provide a desired surface chemistry or to provide additional linkers or linking agents (e.g., for attaching capture agents, polymers, prepolymers, other linkers, or surfaces, such as a surface for a microneedle).

As seen in FIG. 3B, a functionalize nanodiamond 301 can be reacted with one or more prepolymers (e.g., a first prepolymer 310) and capture agents (e.g., that are attached to a linking agent or a prepolymer, as in a responsive prepolymer 320), thereby providing a stimuli-responsive nanodiamond hydrogel. Optionally, the hydrogel can be reacted with a reactive group on a modified wall 330, thereby providing a bound stimuli-responsive hydrogel 350. Additional details regarding optically active particles, reactive groups, functional groups, prepolymers, and capture agents are described herein.

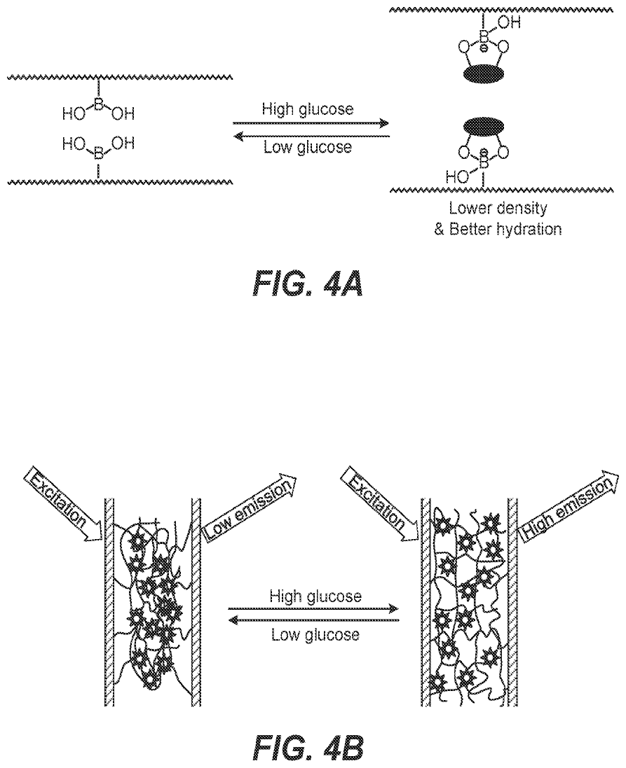

In use, the responsive hydrogel can include any useful capture agent configured to bind or otherwise attach to a target analyte. As seen in FIG. 4A-4B, in one instance, the capture agent includes a boronic acid derivative that binds to glucose. In some embodiments, the hydrogel is functionalized by organotrialkoxysilane with alkene terminal group for covalent integration of fluorescent nano-diamond into a phenylboronic acid functionalized hydrogel.

An example of a responsive hydrogel can include a glucose responsive hydrogel having phenylboronic acid derivatives, a hydrophilic polymer, and fluorescent particles. This hydrogel can provide corresponding light transmission attenuation to the glucose concentration, resulting in a linear response between the glucose concentration and the emission fluorescence light intensity. As glucose molecules diffuse into and out of the hydrogel within the microneedle, they reversibly form 1:1 complex with the phenylboronic acid derivatives. The Donnan osmotic pressure of the hydrogel will increase or decrease. Hence, the hydrogel density, hydration status, and refractive index changes, which causes the changes in light propagation efficiency through the hydrogel. Additional details are provided in the Examples herein.

### Responsive Hydrogels, Including Methods Thereof

The present disclosure encompasses use of a responsive hydrogel, as well as methods of making such hydrogels. In some embodiments, the responsive hydrogel includes a hydrogel backbone (e.g., formed from polymers or prepolymers), one or more optically active particles, and one or more capture agents.

In particular, the hydrogels herein can include any useful polymer. Non-limiting polymers include poly(ethylene oxide) or poly(ethylene glycol) (PEO or PEG), poly(ethylene oxide)-block-poly(acrylic acid) (PEO-b-PAA), poly(ethylene glycol)-co-anhydride, poly(ethylene glycol)-co-lactide, poly(ethylene glycol)-co-glycolide, poly(ethylene glycol)-co-orthoester, poly(propylene oxide) (PPO), poly(2-hydroxyethyl methacrylate) (pHEMA), poly(vinyl alcohol) (PVA), poly(acrylamide) (PAAm), poly(acrylic acid) (PAA), poly(N-isopropylacrylamide) (PNIPAAm), poly(ethylene oxide)-block-poly(N-isopropylacrylamide) (PEO-b-pNIPAAm), poly[poly(ethylene glycol) diacrylate] (p[PEGDA]), poly(acrylamide-co-poly(ethylene glycol) diacrylate) (p[AAM-co-PEGDA]), poly(lactic acid) (PLA), poly(lactic-co-glycolic acid) (PLGA), poly(caprolactone) (PCL), poly(aniline) (PANI), poly(N-(3-amidino)-aniline), poly(octamethylene citric acid), alginate, a poloxamer, poly(dimethylsiloxane), poly(butadiene), poly(isoprene), or a copolymer thereof, as well as prepolymers of any of these.

Non-limiting prepolymers (e.g., monomers or polymeric precursors) include vinyl acetate, ethylene glycol, ethylene oxide, acrylic acid, acrylate, acrylamide, vinyl alcohol, poly(ethylene glycol) divinyl ether, poly(ethylene glycol) diacrylate, and the like. In particular embodiments, the prepolymer includes a vinyl group (e.g., —CH═CH₂), an acrylate group (e.g., —O(CO)—CH═CH₂), a methacrylate group (e.g., —O(CO)—C(CH₃)=CH₂), and ethacrylate group (e.g., —O(CO)—C(CH₂CH₃)=CH₂), and the like. In some embodiments, the vinyl-containing prepolymer includes acrylic acid, allyl amine, styrene, allyl alcohol, acrylamide, acrylate-PEG-hydroxysuccinimde ester, poly(ethylene glycol) diacrylate, vinyl imidazole, vinyl bipyridine, vinyl ferrocene, styrene, pentadiene, methyl pentadiene, or polyacrylated monomer.

Yet other hydrogels include protein hydrogels, such as an albumin hydrogel, avidin hydrogel, lysozyme hydrogel; poly(ethylene glycol) (PEG) macromers (e.g., having vinylsulfone, acrylate, hydroxyl, and/or maleimide reactive groups on branched, multiarm PEG macromers); bifunctional PEGs having reactive groups (e.g., thiol end groups); acrylamides; and the like. Other hydrogel polymeric precursors are described in U.S. Pat. Appl. Pub. Nos. 2010/0055733 and 2015/0144490, each of which is incorporated herein by reference in its entirety.

In particular non-limiting embodiments, a hydrogel refers to a polymeric material that allows a fluid or aqueous medium to diffuse throughout the material. This property of rapid diffusion can allow for rapid contact of the hydrogel and its components with substances dissolved or dispersed within the fluid or aqueous medium. The hydrogel may be polymerized by any technique known to those of ordinary skill in the art, such as, for example, chemical induced polymerization or photopolymerization.

Any components herein can be linked together by use of linking agents, linkers, functional groups, or reactive groups, in which such groups or agents react together to form a bond (e.g., a covalent bond). Components can include, e.g., polymers, prepolymers, particles, capture agents, and/or surfaces of microneedles.

For instance, linking agents can be used to attach a component to a surface. In another instance, linking agents can be used to attach two (or more) components together (e.g., such as in a polymer). In use, a linking agent can react to form a linker between the components. Non-limiting linking agents include compounds including one or more first functional groups, a linker, and one or more second functional groups (e.g., R₁-L-R₂, in which R₁ is the first functional group, L is the linker, and R₂ is the second functional group). In some embodiments, the first functional group allows for linking between a surface and the linker, and the second functional group allows for linking between the linker and the responsive hydrogel or a component thereof (e.g., a capture agent, a particle, a prepolymer, or any agent described herein). In other embodiments, the first functional group allows for linking between a first prepolymer and the linker, and the second functional group allows for linking between the linker and a second prepolymer.

Non-limiting linkers include polyethylene glycol (e.g., —[OCH₂CH₂]ₙ—, in which n is from 1 to 100), an optionally substituted alkane (e.g., an optionally substituted alkylene, as described herein), an optionally substituted heteroalkene (e.g., an optionally substituted heteroalkylene or an optionally substituted alkyleneoxy, as described herein), an optionally substituted carbocyclic ring (e.g., an optionally substituted aromatic ring, such as a phenyl group), optionally substituted heterocyclic ring (e.g., an optionally substituted heteroaryl ring), an optionally substituted aliphatic, an optionally substituted heteroaliphatic, a heteroatom (e.g., silicon, nitrogen, phosphorous, etc.), and the like. In some instances, the linker is covalent bond, such as in a zero-length linker.

Non-limiting functional group can include any useful reactive group, such as halo, hydrogen (H), hydroxyl, optionally substituted hydroxyalkyl, carboxyl, optionally substituted alkenyl (e.g., vinyl), optionally substituted alkynyl, optionally substituted amino, cyano, azido, nitro, thiol, sulfo, optionally substituted alkoxy, optionally substituted aryloxy, optionally substituted aryl, optionally substituted ester, a click chemistry moiety (e.g., an azido group, an alkynyl group, a dienophile group, or a diene group), and the like. A click chemistry moiety can include those from a click-chemistry reaction pair selected from the group consisting of a Huisgen 1,3-dipolar cycloaddition reaction between an alkynyl group and an azido group to form a triazole-containing linker; a Diels-Alder reaction between a diene having a 4π electron system (e.g., an optionally substituted 1,3-unsaturated compound, such as optionally substituted 1,3-butadiene, 1-methoxy-3-trimethylsilyloxy-1,3-butadiene, cyclopentadiene, cyclohexadiene, or furan) and a dienophile or heterodienophile having a 2π electron system (e.g., an optionally substituted alkenyl group or an optionally substituted alkynyl group); a ring opening reaction with a nucleophile and a strained heterocyclyl electrophile; a splint ligation reaction with a phosphorothioate group and an iodo group; and a reductive amination reaction with an aldehyde group and an amino group. Yet other functional groups can include any described herein.

In particular embodiments, the linking agent is a polymerizable compound, where the first functional group includes a vinyl group (e.g., —CH═CH₂), the linker includes an optionally substituted heteroalkylene (e.g., a poly(ethylene glycol) moiety), and the second functional group includes another vinyl group. In this way, the vinyl groups can polymerize with other prepolymer agents having vinyl groups (e.g., in the presence of heat or UV radiation).

In another embodiment, the linking agent is a surface-tethered compound, in which a first functional group is attached to a surface (e.g., of a particle or a microneedle), the linker includes a heteroatom, and the second functional group includes a vinyl group that can polymerize with other prepolymer agents having vinyl group(s).

FIG. 3A shows a particle having various surface-accessible groups, e.g., —C(O)—R_{L1} and/or —R_{L2}, in which each of L1 and L2 is, independently, a leaving group or a reactive group. Non-limiting leaving groups can include H, hydroxyl, halo, amino, alkoxyl, aryloxy, or any other described herein. Such surface-accessible groups can be further modified, such as by way of an agent to differentially functionalize the surface-accessible groups.

As can be seen, a first agent (e.g., a R₁—R_{L3} agent) can selectively react with the carbonyl-containing —C(O)—R_{L1} group, and a second agent (e.g., a R₂—R_{L4}) can selectively react with the —R_{L2} group. In one instance, the first agent (R₁—R_{L3}) is an alkylating group, in which R₁ is an optionally substituted alkyl (e.g., C₁₀₋₅₀ alkyl group) and R_{L3} is a reactive group (e.g., amino, hydroxyl, halo, and the like). The first agent can be used, e.g., to provide adapt the surface characteristics of the particle. The first and second agents can be provided in any useful order (e.g., the first agent can be provided before, after, or at the same time as the second agent).

In another instance, the second agent (R₂—R_{L4}) is a polymerizable group, in which R₂ includes a silyl ether (e.g., —Si(OR)₃, in which each R is, independently, an optionally substituted alkyl), and R_{L4} is a polymerizable group (e.g., alkylene, such as vinyl). In other embodiments, R₂ can include a silanol, a siloxide, a siloxane, a silyl halide, a silyl hydride, a silane, and the like. The second agent can be used, e.g., to polymerize or react with prepolymers. In yet other instances, the second agent is a linking agent (e.g., R_{L4}-L-R_{L5}) having any useful combination of linkers (e.g., L) and functional groups (R_{L4} and R_{L5}), as described herein.

The resulting particle can be a functionalized nanodiamond 301 having different functional groups (e.g., R₁ and R₂ in FIG. 3A), thus allowing the nanodiamond to possess orthogonal chemistry. In addition to nanodiamonds, such an approach can be employed with other particles (e.g., any described herein).

FIG. 3B shows one non-limiting approach to integrate various components within the responsive hydrogel. As can be seen, the functionalize nanodiamond 301 can be provided with a first prepolymer 310 and a responsive prepolymer 320. The first prepolymer 310 can be selected to provide desired properties of the hydrogel polymer, such as desired viscoelastic, absorption, or optical properties. If desired, additional prepolymers can be included to provide a copolymer.

The responsive prepolymer 320 can include functional groups to allow for polymerization, as well as other functional groups that serve as capture agents. In this way, the responsive prepolymer imparts selectivity to bind to certain target analytes, thereby providing stimuli-responsiveness. The capture agent may be provided within the responsive hydrogel during or after polymerization. As can be seen, the first prepolymer 310, the responsive prepolymer 320, and the R₂ reactive group of the functionalized nanodiamond 301 react to form a responsive hydrogel. The amount of each component (as indicated by n1 and n2) can be varied to provide the desired hydrogel.

To allow for attachment to a surface of the microneedle, a modified wall 330 can have a reactive group R₃ that reacts with various groups within the first prepolymer 310 or the responsive prepolymer 320. Upon reaction, the reacted group R₃* can provide a bond that attaches the hydrogel to the microneedle.

While FIG. 3B shows a linear polymer with distinct blocks (e.g., a first block including an n1 number of reacted polymeric portion P1* and a second block including an n2 number of reacted polymeric portion P2*), a skilled artisan would understand that the hydrogel may have a differing configuration based on the initial prepolymer components. For instance, the polymer may be linear or branched, as well as possess any useful block or monomer patterns (e.g., as in block copolymers, alternating copolymers, periodic copolymers, random copolymers, and the like). Furthermore, while only one reacted group R₂* is shown in FIG. 3B, a functionalized nanodiamond can possess multiple surface groups R₂, which can then allow for multiple attachment points within the hydrogel to the nanodiamond.

### Particles

The optically active particles can include those having one or more color centers. Color centers generally include defects within transparent, crystalline insulators or large band-gap semiconductors, such as diamond, silicon carbide, germanosilicate glass, silica, or a perovskite (e.g., LiBaF₃). Such defects can include point defects; substitution defects in which an atom within the substrate is replaced with another atom; and vacancy defects in which an atom is missing within the crystalline lattice, as well as combinations thereof (e.g., nitrogen-vacancy (N—V) centers in diamond having a nitrogen substitution in proximity to a vacancy, nitrogen-vacancy-nitrogen (N-V-N) color centers (or H3 centers) in diamond, germanium-related detects in germanosilicate glass, silicon vacancies silicon carbide, and the like).

In particular embodiments, a plurality of optically active particles has an emission peak from about 450-485 nm or from 500-550 nm. In other embodiments, the optically active particles can include a carbon-based material (e.g., nanodiamonds, carbon nanotubes, carbon nanowires, carbon dots, carbon nanospheres, or carbon particles). In yet other embodiments, the optically active particles can include a metal oxide (e.g., boron oxide, iron oxide, nickel oxide, chromium oxide, zirconium oxide, titanium oxide, silicon oxide, tungsten oxide, manganese oxide, vanadium oxide, copper oxide, zinc oxide, molybdenum oxide, niobium oxide, and natrium oxide).

The particle can be of any useful size. In one embodiment, the particle is a nanoparticle. In another embodiment, the size ranges from about 1 to 10 nm, 1 to 7 nm, or less than 15 nm.

### Capture Agents

Capture agents can be used to bind to an analyte or detect a condition. Non-limiting analytes can include an ion, a small molecule, a metal (e.g., a metal ion or a metal atom), a particle (e.g., a metal particle or a magnetic particle), a peptide, a protein, a cytokine, a hydrophilic sample, or a hydrophobic sample (e.g., oil, gasoline, and the like). Non-limiting conditions can include a temperature, an ionic state, a hydration level, and the like.

Capture agents can include any useful functional groups that allows for capture an analyte or detecting a condition. Such functional groups can include boronic acids or boronate groups (e.g., a compound having a —B(OH)₂ group), Schiff bases (e.g., an imine), acrylic acids, amides, amines, thiols, ionizable groups (e.g., weakly acidic or basic groups, such as sulfonate, carboxylate, and/or quaternary ammonium groups), reducible groups (e.g., disulfide groups), charged groups (e.g., anionic or cationic groups), chelating groups (e.g., an aminopolycarboxylic acid, carboxylic acids, crown ethers, and the like), particles (e.g., magnetic particles), temperature responsive groups, redox indicators (e.g., having one or more metal complexes that provide an optical change in response to changes in oxidation states; or having one or more organic groups that provide an optical change in response to changes in oxidation states, such as those present in ring systems, such as MB (methylene blue) or ABTS (2,2′-azino-bis(3-ethylbenzothiazoline-6-sulfonic acid)), photosensitizers (e.g., configured to provide singlet oxygen or an electron or to abstract a hydrogen atom upon exposure to light), dyes, antibodies, nanostructures (e.g., nanofilaments, such as silicon nanofilaments), or hydrophobic groups (e.g., optionally substituted C₈₋₅₀ alkyl groups). Non-limiting analytes can include glucose, lactate, uric acid, glutathione, carbon dioxide, a peroxide (e.g., hydrogen peroxide), and the like.

In particular embodiments, the capture agent includes a boronic acid or a boronate group, such as —Ar—B(OH)₂ or -Ak-B(OH)₂, in which Ar is an optionally substituted aryl or aromatic; and in which Ak is an optionally substituted alkylene, heteroalkylene, aliphatic, or heteroaliphatic.

In other embodiments, the capture agent includes a redox indicator group, such as an optionally substituted phenothiazinyl group or an optionally substituted phenothiazinylidene group. The phenothiazinyl or phenothiazinylidene group can be substituted with any group described herein, e.g., such as for alkyl. In particular embodiments, phenothiazinyl or phenothiazinylidene is optionally substituted with one or more optionally substituted amino (e.g., —NR^{N1}R^{N2}, where each of R^{N1} and R^{N2} is, independently, H, optionally substituted alkyl, or optionally substituted aryl, or R^{N1} and R^{N2}, taken together with the nitrogen atom to which each are attached, form a heterocyclyl group, as defined herein); optionally substituted azanium (e.g., =NR^{N1}R^{N2}, where each of R^{N1} and R^{N2} is, independently, H, optionally substituted alkyl, or optionally substituted aryl, or R^{N1} and R^{N2}, taken together with the nitrogen atom to which each are attached, form a heterocyclyl group, as defined herein); or optionally substituted alkyl. The nitrogen and/or sulfur atoms present in the phenothiazinyl or phenothiazinylidene group can have any useful charge, valency (e.g., divalent, trivalent, tetravalent, etc.), or types of bonds (e.g., single, double, or triple bonds). The phenothiazinyl or phenothiazinylidene group can also include one or more salts (e.g., ionic salts), such as any described herein.

Any useful capture agent can be used in combination with the present disclosure. The capture agent can directly or indirectly bind the analyte of interest. Further, multiple capture agents can be used to bind the analyte and provide a detectable signal for such binding. For instance, multiple capture agents are used for a sandwich assay, which requires at least two capture agents.

Non-limiting capture agents include one or more of the following: a protein that binds to or detects one or more analytes (e.g., an antibody or an enzyme), a globulin protein (e.g., bovine serum albumin), a peptide, a nucleotide, a particle, a microparticle, a sandwich assay reagent, a catalyst (e.g., that reacts with one or more analytes), and/or an enzyme (e.g., that reacts with one or more analytes, such as any described herein).

The present device can be used to determine any useful target analyte or condition. Non-limiting analytes include one or more physiologically relevant markers, such as glucose, lactate, pH, a protein (e.g., myoglobin, troponin, insulin, or C-reactive protein), an enzyme (e.g., creatine kinase), a catecholamine (e.g., dopamine, epinephrine, or norepinephrine), a cytokine (e.g., TNF-α or interleukins, such as IL-6, IL-12, or IL-10), an antibody (e.g., immunoglobulins, such as IgA), a biomolecule (e.g., cholesterol or glucose), a neurotransmitter (e.g., acetylcholine, glutamate, dopamine, epinephrine, neuropeptide Y, or norepinephrine), a signaling molecule (e.g., nitric oxide), an antigen (e.g., CD3, CD4, or CD8), an ion (e.g., a cation, such as K⁺, Na⁺, H⁺, or Ca²⁺, or an anion, such as Cl⁻ or HCO₃⁻), carbon dioxide (CO₂), oxygen (O₂), hydrogen peroxide (H₂O₂), a cancer biomarker (e.g., human ferritin, carcinoembryonic antigen (CEA), prostate serum antigen, human chorionic gonadotropin (hCG), diphtheria antigen, or C-reactive protein (CRP)), a hormone (e.g., hCG, epinephrine, testosterone, human growth hormone, epinephrine (adrenaline), thyroid hormone (e.g., thyroid-stimulating hormone (TSH), thyroxine (TT4), triiodothyronine (TT3), free thyroxine (FT4), and free triiodothyronine (FT3)), adrenal hormone (e.g., adrenocorticotrophic hormone (ACTH), cortical hormone (F), and 24-hour urine-free cortisol (UFC)), a gonadal hormone (e.g., luteinizing hormone (LH), follicle-stimulating hormone (FSH), testosterone, estradiol (E2), and prolactin (PRL)), cortisol, leptin, or a peptide hormone, such as insulin), an inflammatory marker (e.g., CRP), a disease-state marker (e.g., glycated hemoglobin for diabetes), a cardiovascular marker (e.g., CRP, D-dimer, troponin I or T), a blood marker (e.g., hematocrit or hemoglobin, including hemoglobin A1c), a cell (e.g., a leukocyte, neutrophil, B-cell, T-cell, lymphocyte, or erythrocyte), a viral marker (e.g., a marker for human immunodeficiency virus, hepatitis, influenza, or chlamydia), a metabolite (e.g., glucose, cholesterol, triglyceride, creatinine, lactate, ammonia, ascorbic acid, peroxide, potassium, glutamine, or urea), a nucleic acid (e.g., DNA and/or RNA for detecting one or more alleles, pathogens, single nucleotide polymorphisms, mutations, etc.), an amino acid (e.g., glutamine), a drug (e.g., a diuretic, a steroid, a growth hormone, a stimulant, a narcotic, an opiate, etc.), etc. Other non-limiting markers include one or more pathogens, such as Mycobacterium tuberculosis, Diphtheria antigen, Vibrio cholera, Streptococcus (e.g., group A), etc.

### Microneedles

The microneedle can, in some instances, include an optically transparent material to allow for sufficient detection of the optical output signal from the particles within the hydrogel. In further instances, the microneedle can include a porous material, which can increase diffusion of ISF or analytes from the ISF into the hydrogel. Non-limiting materials for the microneedle can include, e.g., glass, sapphire, diamond, ruby, silica, polycarbonate, poly(dimethylsiloxane), poly(vinyl chloride), poly(methyl methacrylate), polyethylene, and combinations thereof.

A surface of the microneedle (e.g., inner surface within the bore or outer surface of the microneedle) can be modified or surface treated to allow for further functionalization. For instance, the inner surface within the bore can be modified to allow for covalent binding to the responsive hydrogel. In another instance, the outer surface of the microneedle can be modified to attach a biocompatible coating.

Within the device, one or more microneedles may be employed. In some instances, the device can include a plurality of microneedles, wherein each of the plurality of microneedles includes an internal bore and a hydrogel disposed herein. Use of more than one microneedle can be useful to increase sample access. The microneedle(s) can be configured to obtain a sample including ISF from a subject, such as by having a coating, a puncturing edge, a particular length to optimize penetration depth, and the like.

Such coatings can include an anti-inflammatory coating, an anti-immunogenic coating, or a biocompatible coating (e.g., a polymeric coating, a metal coating, a perfluorinated coating, and the like). Non-limiting coatings include a hydrogel, a polyether (e.g., a polyethylene glycol or a polypropylene glycol), a polymer (e.g., an epoxy, a polyaniline), a dendrimer, a metal (e.g., a noble metal, such as gold, platinum, silver, etc.), an oxide coating (e.g., a zirconium oxide, a tin oxide, a zinc oxide, or a titanium oxide coating, including other dopants such as silicon, barium, manganese, iron, etc., such those coatings obtained by atomic layer deposition, hydrothermal conversion, sol-gel conversion, thermal annealing, and/or thermal evaporation), a ceramic (e.g., boron nitride), etc.

In some embodiments, each needle has an interior surface facing the hollow bore and an exterior surface, the distal end of the exterior surface for at least one needle includes a puncturing edge, and at least one needle has a length of more than about 0.5 mm or from about 0.1 mm to about 7 mm (e.g., from 0.1 mm to 0.5 mm, 0.1 mm to 1 mm, 0.1 mm to 1.5 mm, 0.1 mm to 2 mm, 0.1 mm to 2.5 mm, 0.1 mm to 3 mm, 0.1 mm to 3.5 mm, 0.1 mm to 4 mm, 0.1 mm to 4.5 mm, 0.1 mm to 5 mm, 0.2 mm to 0.5 mm, 0.2 mm to 1 mm, 0.2 mm to 1.5 mm, 0.2 mm to 2 mm, 0.2 mm to 2.5 mm, 0.2 mm to 3 mm, 0.2 mm to 3.5 mm, 0.2 mm to 4 mm, 0.2 mm to 4.5 mm, 0.2 mm to 5 mm, 0.2 mm to 7 mm, 0.3 mm to 0.5 mm, 0.3 mm to 1 mm, 0.3 mm to 1.5 mm, 0.3 mm to 2 mm, 0.3 mm to 2.5 mm, 0.3 mm to 3 mm, 0.3 mm to 3.5 mm, 0.3 mm to 4 mm, 0.3 mm to 4.5 mm, 0.3 mm to 5 mm, 0.3 mm to 7 mm, 0.5 mm to 1 mm, 0.5 mm to 1.5 mm, 0.5 mm to 2 mm, 0.5 mm to 2.5 mm, 0.5 mm to 3 mm, 0.5 mm to 3.5 mm, 0.5 mm to 4 mm, 0.5 mm to 4.5 mm, 0.5 mm to 5 mm, 0.5 mm to 7 mm, 0.7 mm to 1 mm, 0.7 mm to 1.5 mm, 0.7 mm to 2 mm, 0.7 mm to 2.5 mm, 0.7 mm to 3 mm, 0.7 mm to 3.5 mm, 0.7 mm to 4 mm, 0.7 mm to 4.5 mm, 0.7 mm to 5 mm, 0.7 mm to 7 mm, 1 mm to 1.5 mm, 1 mm to 2 mm, 1 mm to 2.5 mm, 1 mm to 3 mm, 1 mm to 3.5 mm, 1 mm to 4 mm, 1 mm to 4.5 mm, 1 mm to 5 mm, 1 mm to 7 mm, 1.5 mm to 2 mm, 1.5 mm to 2.5 mm, 1.5 mm to 3 mm, 1.5 mm to 3.5 mm, 1.5 mm to 4 mm, 1.5 mm to 4.5 mm, 1.5 mm to 5 mm, 1.5 mm to 7 mm, 3 mm to 3.5 mm, 3 mm to 4 mm, 3 mm to 4.5 mm, 3 mm to 5 mm, and 3 mm to 7 mm). In some embodiments, a plurality of microneedles is provided in an array (e.g., two, three, four, five, six, seven, eight, nine, ten, fifteen, twenty, or more needles in array).

The device herein can have one or more needles of any useful dimension, such as length, width, height, circumference, and/or cross-sectional dimension. In particular, a skilled artisan would be able to optimize the needle length based on the type of fluid or type of tissue to be measured.

The needles can be formed from any useful material, e.g., glass, a polymer (e.g., a biocompatible polymer or an acrylate-based polymer), a ceramic, a composite material, etc. The surface (e.g., interior and/or exterior surface) of the needle can be surface-modified with any agent described herein (e.g., a linking agent, capture agent, label, and/or porous material, as described herein). Additional surface-modified needles are described in U.S. Pat. Pub. No. 2011/0224515, as well as U.S. Pat. Nos. 7,344,499 and 6,908,453, each of which is incorporated by reference herein in its entirety.

The needles can be formed from any useful process. For instance, when formed from a polymer, the needle can be formed by polymerizing, molding (e.g., melt-molding), spinning, depositing, casting (e.g., melt-casting), etc. Methods of making needles are described in U.S. Pat. Nos. 7,344,499 and 6,908,453, each of which is incorporated by reference herein in its entirety. In an embodiment, porogen leaching technology may be utilized to fabricate microneedles of a porous hollow structure.

In some embodiments, a plurality of hollow needles is configured to obtain the sample from a subject. In particular embodiments, at least one needle includes a puncturing edge (e.g., a tapered point, a sharpened bevel, or one or more prongs). Furthermore, a plurality of needles can be provided in an array. The array can include two, three, four, five, six, seven, eight, nine, ten, fifteen, twenty, or more needles configured in any useful arrangement (e.g., geometrical arrangements). The array can have any useful spatial distribution of needles (e.g., a square, rectangular, circular, or triangular array), a random distribution, or the like. In some instances, each needle within the array is individually optically addressable.

In some instance, the microneedle (or a plurality of microneedles) extends from a planar substrate. The planar substrate, in some examples, can be a flexible substrate, which can be useful for affixing to a skin surface of a subject. Non-limiting materials for a flexible substrate includes poly(dimethylsiloxane), poly(caprolactone), poly(lactic acid), or natural rubber (e.g., manufactured by casting, 3D printing, laser sintering, laser etching, and the like).

In general, a substrate refers to a substantially planar surface or media containing one or more structures. For instance, one or more needles, fluidic channels, optical relay components, optical sensors, optical detectors, controllers, transmitters, and/or other components component can be embedded in the same substrate or in different substrates. The substrate can be formed from any useful material. Non-limiting materials include any described herein, such as a flexible substrate (e.g., a poly(vinyl acetate), a poly(ester), or any other described herein) or a printed circuit board (PCB).

The substrate can include one or more inlets (or vias) in fluidic communication with the needle. In this manner, a sample collected within the needle can be delivered through the needle and into the inlet. Generally, the inlet is further configured to be in fluidic communication with one or more fluidic channels, which can be used to store or deliver a fluid. Other structures can be integrated into a substrate, such as, e.g., a filter, a permeable or semi-permeable membrane, a valve, and/or an electrode.

### Additional Components, Including Devices and Systems Having Such Components

The devices and systems herein can include one or more components, which may optionally be provided separately or integrated together (e.g., in a monolithic structure).

Non-limiting components can include an optical source, such as a light emitting diode (LED), a laser, and the like; an optical sensor, such as a photodiode; or a wireless signal transmitter, such as a Bluetooth antenna. Other components can include an optical relay component, such as an optical waveguide, an optical fiber, or a pair of mirrors, as well as other passive optical elements or even active optical elements. Waveguides can have any useful configuration, such as ridge waveguides, coaxial waveguides, rectangular waveguides, slab waveguides, planar waveguides, channel waveguides, etc. In some embodiments, optical waveguides can be formed from a polymer or a semiconductor material (e.g., a III-V material, such as InP).

The device or system can include a controller, which can communicate with hardware components. In one instance, the controller can include a memory and a processor, which can be configured to be electrically connected to the optical source and the optical sensor. Furthermore, the processor and the memory can be communicatively connected with one another, in which the processor is at least operatively connected with hardware (e.g., the optical source, optical sensor, and/or wireless transmitter), and the memory stores computer-executable instructions for controlling the processor to at least control the hardware by: (a) transmitting an optical input signal from the optical source to the microneedle, thereby allowing the optical input signal to be transmitted through the hydrogel disposed within the microneedle; and (b) receiving an optical output signal from the microneedle to the optical sensor, wherein the optical output signal is indicative of a presence or an absence of an analyte captured within the hydrogel. In particular embodiments, such instructions can also include: (c) obtaining data from the optical sensor indicating the presence or the absence of the analyte; and (d) storing data within the memory and/or transmitting the data to the wireless signal transmitter.

The controller may include one or more memory devices, one or more mass storage devices, and one or more processors. The processor may include a CPU or computer, analog, and/or digital input/output connections, controller boards, etc. A controller may execute system control software stored in a mass storage device, loaded into a memory device, and executed on a processor. Alternatively, the control logic may be hard coded in the controller. Applications Specific Integrated Circuits, Programmable Logic Devices (e.g., field-programmable gate arrays, or FPGAs) and the like may be used for these purposes. Herein, wherever “software” or “code” is used, functionally comparable hard coded logic may be used in its place.

The apparatuses and methods described herein may be implemented by one or more computer programs executed by one or more processors. The computer programs include processor-executable instructions that are stored on non-transitory computer readable medium. The computer programs may also include stored data. Non-limiting examples of the non-transitory tangible computer readable medium are non-volatile memory, magnetic storage and optical storage.

The device or system can include one or more fluidic channels (including inlets), chambers, reservoirs, and the like can be used to effect fluidic communication between two structures or regions. One or more optical relay components may also be used to effect optical communication between optical components, such as optical sensors, detectors, or microneedles.

Any of the microneedles, fluidic channels, chambers, reservoirs, optical components, and substrates described herein can be surface modified (e.g., to increase biocompatibility, decrease protein adsorption or absorption, and/or decrease surface contamination).

In some embodiments, the system can be a monitoring system including a sampling component and a detecting component, as well as optical relay component(s) to provide optical communication between the sampling and detecting components. The sampling component can include a microneedle having a responsive hydrogel (e.g., any describe herein). The detecting component can include an optical source configured to transmit an optical input signal to the microneedle; an optical sensor configured to receive an optical output signal from the microneedle; and a controller configured to be electrically connected to the optical source and the optical sensor. The monitoring system can further include a communicating component, which includes a wireless signal transmitter configured to transmit data from the controller or the memory to an external receiver.

In some embodiments, the system can include a fluidic component including one or more fluidic channels configured for fluidic communication with at least one microneedle. Such a fluidic component can be used to deliver or remove fluidic within the microneedle. The fluidic component can optionally include a pump.

Yet other non-limiting components include a transducer (e.g., an electrode or an array of electrodes); a membrane (e.g., placed between the needle and the channel; placed within a channel, such as to filter one or more particles within the sample; and/or placed within a needle); a multifunctional sensor (e.g., to measure temperature, strain, and electrophysiological signals, such as by using amplified sensor electrodes that incorporate silicon metal oxide semiconductor field effect transistors (MOSFETs), a feedback resistor, and a sensor electrode in any useful design, such as a filamentary serpentine design); a microscale light-emitting diode (LEDs, which can be used as an optical source); an active/passive circuit element (e.g., such as transistors, diodes, and resistors); an actuator; a wireless power coil; a device for radio frequency (RF) communications (e.g., such as high-frequency inductors, capacitors, oscillators, and antennae); a resistance-based temperature sensor; an optical filter; a photodetector; a photovoltaic cell; and a diode, such as any described in Kim D H et al., “Epidermal electronics,” Science 2011; 333:838-43, which is incorporated herein by reference. These components can be made from any useful material, such as, e.g., silicon and gallium arsenide, in the form of filamentary serpentine nanoribbons, micromembranes, and/or nanomembranes.

The device can further include a power source to operate the controller, optical source, and/or optical detector. In particular embodiments, the device includes a data-processing circuit powered by the power source and electrically connected to the optical source and/or optical detector. In further embodiments, the device includes a data output port for the data-processing circuit.

The present disclosure can be useful for autonomous remote monitoring of a subject. The device can be placed on the skin of a subject, and the presence or absence of one or more analytes can be remotely relayed to a heath care worker. Accordingly, the device described herein can include one or more components that would allow for such relay. Non-limiting components include an analog-to-digital converter, a radiofrequency module, and/or a telemetry unit (e.g., configured to receive processed data from a data-processing circuit electrically connected to the transducer and to transmit the data wirelessly). In various embodiments, the telemetry unit is fixed within the platform or packaged separately from the platform and connected thereto by a cable.

### Methods Thereof

Methods of using the devices and systems herein are also envisioned. Such methods can include detecting an analyte (e.g., any herein), such as, e.g., for the treatment of diabetes. Methods can include applying a microneedle to a target site of a subject; and measuring one or more optical output signals transmitted from the microneedle, wherein the one or more optical output signals are indicative of a presence or an absence of the analyte captured within the hydrogel the target site is a dermal surface of the subject.

Applying can include providing access to ISF at the target site of the subject. Such applying can include affixing the microneedle at the target site for a period of about one to six months. For such long-term use, the microneedle can optionally include an anti-inflammatory coating, an anti-immunogenic coating, or a biocompatible coating. Any of the surfaces described herein may be modified to promote biocompatibility, to functionalize a surface (e.g., using one or more capture agents including the optional use of any linking agent), or both. The surface can be modified with any useful agent, such as any described herein. Non-limiting agents include a capture agent (e.g., any described herein); a polymer, such as a conducting polymer (e.g., poly(pyrrole), poly(aniline), poly(3-octylthiophene), or poly(thiophene)), an antifouling polymer, a biocompatible polymer (e.g., chitosan), or a cationic polymer; a coating; a film; a linking agent (e.g., any described herein); an enzyme, such as glucose oxidase, cholesterol oxidase, horseradish peroxidase, or any enzyme useful for oxidizing, reducing, and/or reacting with an analyte of interest; or combinations thereof.

Detecting an analyte can include measuring or detecting an optical emission intensity having a wavelength of about 450-485 nm. Further methods can include: analyzing the one or more optical output signals to determine the presence or the absence of the analyte, thereby providing processed data; and transmitting the processed data to an external receiver.

The present device can be applied for any useful method and/or adapted for any particular use. For instance, point-of-care (POC) diagnostics allow for portable systems, and the device herein can be adapted for POC use. In some embodiments, the device for POC use includes a microfluidic processing structure (e.g., any structure described herein, such as a needle, a substrate, and/or a channel), a target recognition region (e.g., including an optical source, an optical sensor, and one or more optical relay components, as described herein), and/or an electronic output (e.g., including a controller). Non-limiting POC devices and uses are described in Gubala V et al., “Point of care diagnostics: status and future,” Anal. Chem. 2012; 84(2):487-515, which is incorporated by reference in its entirety. Such POC devices can be useful for detecting one or more analytes for patient care, drug and food safety, pathogen detection, diagnostics, etc.

Wearable sensors can allow for minimally invasive monitoring of physiological functions and elimination of biological fluid transfer between subject and device; these devices can be capable of providing real-time analysis of a patient's condition. In other embodiments, the device is adapted to include one or more components allowing for a wearable sensor. Such components include a telemetry network including one or more devices (e.g., as described herein) and one or more flexible substrates (e.g., including cloth, plastic, or fabric, e.g., Gore-Tex™, an expanded polytetrafluoroethylene (ePTFE), polyimide, polyethylene naphthalate, polyethylene terephthalate, biaxially-oriented polyethylene terephthalate (e.g., Mylarm), or PTFE).

### EXAMPLES

The following examples are included to demonstrate non-limiting embodiments of the disclosure. It should be appreciated by those of skill in the art that the techniques disclosed in the examples that follow represent techniques that could be modified. For instance, those of skill in the art could, in light of the present disclosure, appreciate that many changes can be made in the specific embodiments that are disclosed and still obtain a like or similar result without departing from the spirit and scope of the disclosure.

### Example 1: Continuous Monitoring of Glucose with Nano-Diamond Hydrogel in Microneedles

Continuous, non-invasive monitoring of glucose level for diabetes patients remains a challenge. Currently, most of the continuous monitoring sensors for glucose are enzyme electrodes or microdialysis probes implanted under the skin. These sensors usually require oxygen for activity, are insufficiently stable in vivo, and exhibit poor accuracy under the low glucose conditions.

The presence of interfering electroactive substances in tissues can also cause impaired responses and signal drift in vivo, which necessitate frequent calibrations of current sensors. A fluorescence-based glucose sensor in the skin will likely be more stable, have improved sensitivity, and resolve the issue of electrochemical interference from the tissue. However, traditional fluorescence molecules usually have poor photostability, and are not suitable for long term imaging.

Herein, we describe a non-limiting nano-diamond hydrogel system, which has superior photostability and biocompatibility. Coupled with a microneedle system, this technology could provide a non-invasive and long term glucose monitoring platform for diabetes patients. As such a system could be adapted to detect glucose or other analytes, monitoring of various disease states or conditions is envisioned. Additional details are described in the following Examples.

### Example 2: Microneedle with Glucose-Responsive Hydrogel for Continuous Blood Glucose Monitoring

The diabetic population is rapidly increasing and predicted to reach 366 million by 2030, which is a global health threat that poses a devastating impact on society.¹⁻³ Considering the prevention of diabetic complications to the heart, kidney, retina, and neural system, it is crucial to maintain a normal blood glucose concentration.⁴ Currently, the fingertip prick method is used to collect a blood sample, and this method is most commonly used to accurately detect glucose concentration in blood. Nonetheless, at least three or four finger stick blood tests per day must be performed, which presents discomfort to patients.

For decades, aiming at in vivo glucose monitoring with less effort by patients and less tissue damage, fully-implantable glucose sensors such as microdialysis, enzyme-tipped catheters, and genetically engineered skin grafts have been developed.⁵⁻⁸ However, these methods have several drawbacks for long-term use, e.g., such as insufficient stability in vivo, poor precision, and oxygen-dependent activity. Clark et al. have designed a fluorescent sensor based on ion-selective optodes capable of detecting small molecules.⁹ The sensor is based on entrapping octylboronic acid and alizarin within a lipophilic core, which is formed by self-assembly of polymers. However, because of unavoidable diffusion and gradual leaching, this sensor is typically stable only for a few hours.

Described herein is a wearable microneedle device for continuous blood glucose monitoring. In particular embodiments, the device can include a light source and an optical sensor on a mini size chip with a wireless signal transmitter (see, e.g., FIG. 1B). Typically, at least two optical fibers are used: a first optical fiber to transmit an excitation light from the light source to the microneedle; and a second optical fiber to transmit an emission light from the microneedle to the optical sensor. A transparent microneedle with a glucose responsive hydrogel was used to test the blood glucose with responsive optical intensity. This painless device can effectively monitor the long-term change of blood glucose.

The microneedle can be configured to detect glucose by selectively binding to glucose and then emitting an optical signal indicative to such binding. In particular embodiments, the microneedle is a hollow transparent microneedle having an inner surface that is modified and cross-linked with a glucose responsive hydrogel. This hydrogel exhibits corresponding light transmission attenuation to the glucose concentration, resulting in a linear response between the glucose concentration and the light intensity.

With a minimally invasive prick, interstitial fluid from the skin will flow into the inner hydrogel of the microneedle. After irradiation by light from the first optical fiber (attached to a light source, such as a light emitting diode), the hydrogel can responsively emit light with an intensity that corresponds to the glucose concentration. The second optical fiber is then employed to capture light emitted from the microneedle and transmit it to the optical sensor. A wireless signal transmitter (e.g., a Bluetooth antenna) can then transfer data (e.g., a signal) from the sensor to an external receiver (e.g., a cellphone or laptop) to show the corresponding blood glucose profile, glucose concentration, light intensity, or other data.

### Example 3: Non-Limiting Microneedle with Blue Fluorescent Nanodiamond Hydrogel

Fluorescent nanodiamonds have attracted much attention for biomedical applications because they show low or no cytotoxicity, good photostability, and high quantum yield.¹⁰⁻¹³ Described herein is a non-limiting blue fluorescent hydrogel composed of acrylamide, 3-(acrylamido)phenylboronic acid, poly(ethylene glycol) diacrylate, and vinyl modified fluorescent nanodiamond. This hydrogel has lower light transmission attenuation in a glucose-rich solution as compared to a low glucose solution, resulting in a linear fluorescence response between the glucose concentration and the emission light intensity (FIG. 4A-4B). A hollow glass microneedle possessed an inner surface modified with a vinyl group, which was then cross-linked with the fluorescent hydrogel. Non-limiting methods of preparing such microneedles are described below.

Synthesis of blue fluorescent nanodiamond: The fluorescent nanodiamond was synthesized using the wet chemistry method.¹⁴ As shown in FIG. 6, 30 mg of detonation nanodiamond was purified by air oxidation and cleansed of metal impurities by reflux with 35 wt % hydrochloric acid for a day. After refluxing with 50 mL of thionyl chloride and 1 mL of anhydrous dimethylformamide at 70° C. for 24 hours, the solid was washed with anhydrous tetrahydrofuran (twice) and then dried at room temperature under vacuum. The solid was stirred in a sealed flask with 1 g of octadecylamine at 90-100° C. for 96 hours. After ultrasonication and washes with anhydrous methanol (five times) to removed excess octadecylamine, the product was reflux with 10 M sodium hydroxide for three hours. The solid was wash (three times) with water and then stirred with 0.03 mL of triethoxyvinylsilane, 1.9 mL of ethanol, and 0.07 mL of water for three hours. At last, the synthesized blue fluorescence nanodiamond was collected after washed with ethanol (three times) and then vacuum dried.

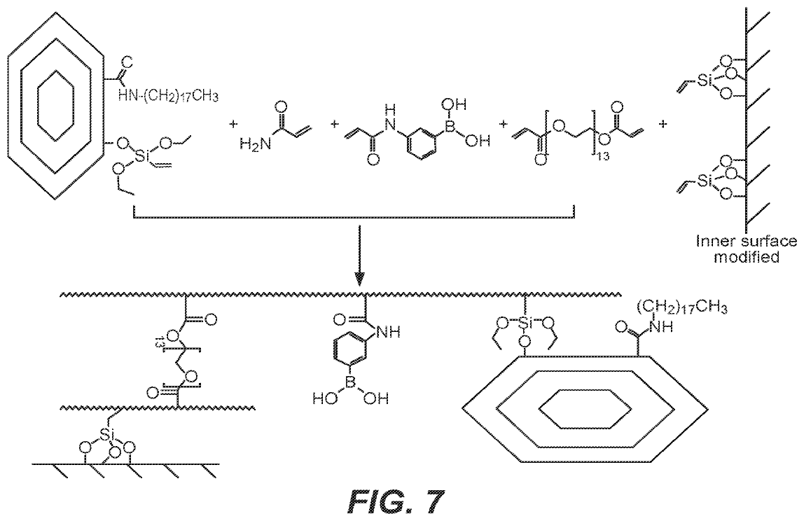

Preparation of responsive nanodiamond hydrogel in glass microneedle: As shown in FIG. 7, the inner surface of the glass microneedle was first cleaned and then treated with a solution containing triethoxyvinylsilane, ethanol, and water (3: 190: 7 vol. %) overnight. The glucose-responsive hydrogel was synthesized as a phenylboronic acid-functionalized polyacrylamide gel.^{15,16} About 156 mg of acrylamide, 76.4 mg of 3-(acrylamido)phenylboronic acid, and 56 mg of poly(ethylene glycol) diacrylate were dissolved in a suspension containing 0.1 wt. % blue fluorescence nanodiamond water by ultrasonication. About 10 μl of N,N,N′,N′-tetramethylethylenediamine was added to the suspension and then degassed. About 200 μl of the suspension was mixed with 100 μl of 40 mg/ml fresh ammonium persulfate solution. One end of the glass microneedle was immediately put into the mixed suspension. After the hollow chamber of the microneedle was filled, the microneedle was taken out to allow formation of the hydrogel within the microneedle. The microneedle with the responsive nanodiamond hydrogel was then incubated in distilled water overnight to wash out unreacted molecules. After that, it was stored in 1×sodium phosphate buffer solution.

### Example 4: Fluorescence Response to Glucose In Vitro

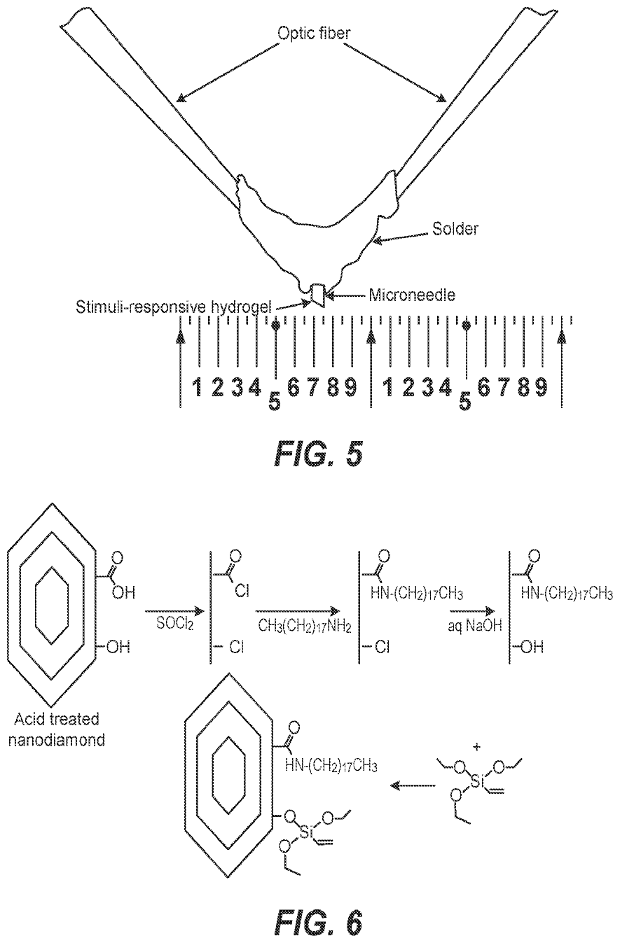

Microneedles were prepared, as described in Example 3. After assembling the device as indicated in FIG. 1B and FIG. 5, the sharp tip of the microneedle was immersed into a series of sodium phosphate buffer solutions with different glucose concentrations (0-500 mg/dl). Then, the microneedle was exposed to an excitation light source, which was generated by a high power LED chip (UV 365 nm, 600 mA, 5 W; Chanzon). The emission light intensity was recorded within 10 minutes, as soon as a constant optical intensity had been reached. Emission light was monitored at 450 nm by an on-chip multispectral sensor (400-1000 nm, pixel sensor; Ocean Optics).

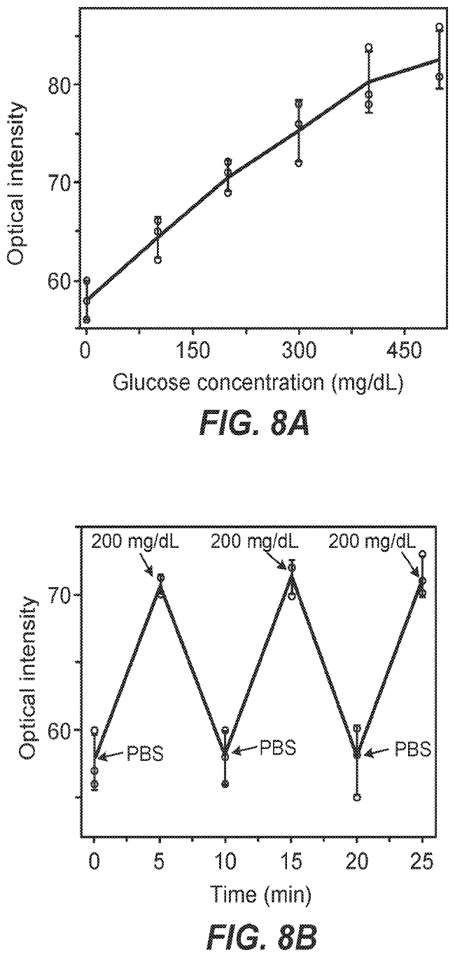

FIG. 8A shows a linear response between emission intensity (from light being emitted from the microneedle) and glucose concentration. A higher concentration of glucose resulted in an increase in emission intensity.

Without wishing to be limited by mechanism, turbidity within the hydrogel generally arises from microheterogeneities, which mostly originates from the association of the polymer chains due to hydrophobic interactions between the neutral phenylboronic acid groups. As glucose molecules diffuse into the hydrogel, a complex forms between the phenylboronic acid groups and glucose. The formation of this complex within the hydrogel, in turn, results in shifting the pKa of the phenylboronic acid groups to lower values, increasing the hydrophilicity of the polymer chains, and decreasing the association between the polymer chain groups. In addition, the Donnan osmotic pressure of the hydrogel will also increase. Hence, upon binding glucose, physical characteristics of the hydrogel changes (e.g., characteristics such as hydrogel density and refractive index), thereby causing an increase in light propagation efficiency. Accordingly, as binding increases between the capture agent (here, phenylboronic acid or boronic acid group) and the analyte (here, glucose), emission of optical signals through the hydrogel increases. The optical signals arise from the optically active particles (here, blue nanodiamonds) that are trapped within the hydrogel.

The observed effects are reversible, thereby allowing the same microneedle with responsive hydrogel to be used more than once. For instance, reversible complex formation of the phenylboronic acid groups and cis diols of the glucose molecules could enable the application of the microneedle as dynamic sensor. FIG. 8B illustrates a non-limiting sensor. As can be seen, as the concentration of glucose was decreased, the emission intensity returned to the original intensity. The complex formation/deformation time was around 5 minutes for each glucose concentration change.

### Example 5: Fluorescence Response to Glucose In Vivo

In vivo experiments were conducted. In particular, the in vivo glucose response was characterized using male mouse (CD1, 9-week-old). The mouse was anesthetized by isoflurane (Forane®, Abbott) of 1.5 mL/h delivered via a Univentor 400 (Univentor Ltd.). The body temperature of the mouse was kept at 37° C. during the experiment, and the mouse was fasted overnight (12 h) before the experiment. To increase blood glucose concentrations, we injected 10 wt. % glucose solution (1.5 g/kg glucose/body weight) into mouse through an intraperitoneal injection. After that, we measured blood glucose concentrations using a standard glucose meter (EZ meter; Bayer Contour NEXT) by blood sample from the snipped tail and record the emission light intensity every 10 minutes.

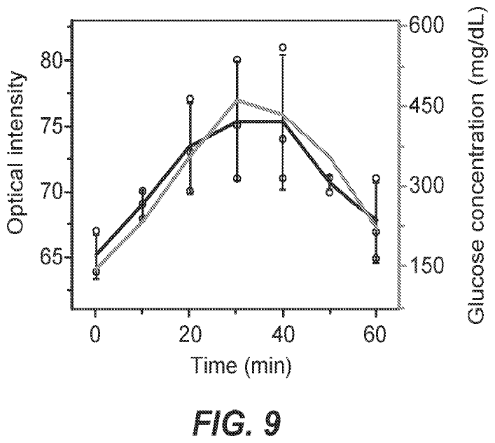

As shown in FIG. 9, we experimentally verified the correlation between fluorescence intensity and in vivo blood glucose concentration. We injected glucose to temporally elevate the blood glucose to around 450 mg/dl, which then slowly decreased to around 225 mg/dl in blood. As can be seen, the observed emission intensity was consistent with the measured fluctuation in blood glucose concentration.

### Example 6: In Vivo Mouse Study of Blood Glucose Level Monitoring

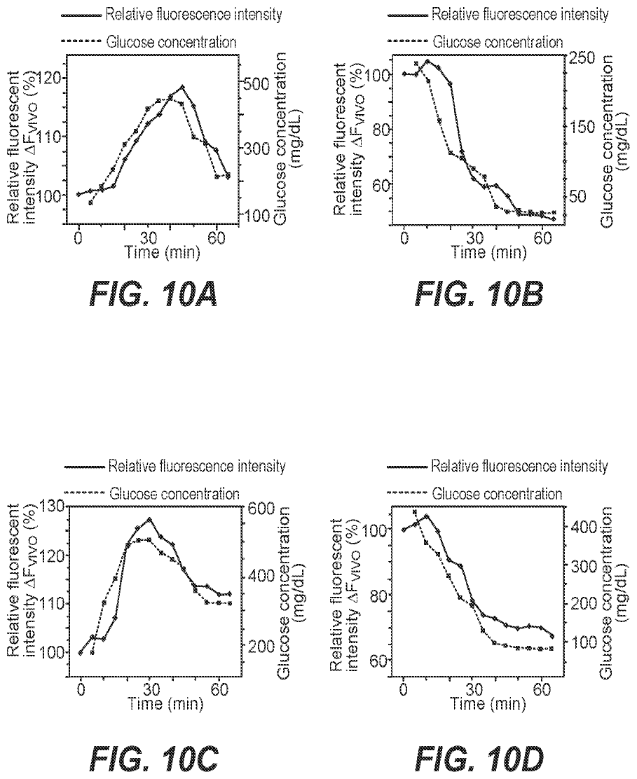

To test the glucose sensing capability in vivo, we mounted the microneedle device having porous microneedles with transparent barrels filled with fluorescent nano-diamond based boronic hydrogel prepared in accordance with Example 3 to mouse skin and carried out the IPGTT (intraperitoneal glucose tolerance test). Fasted animals were administered with a bolus of glucose intraperitoneally. The blood glucose level was determined by the nano-diamond hydrogel and a commercial glucose monitoring system (Bayer Contour) with blood samples taken from snipped tail simultaneously. FIG. 10A shows the correlation between the measured glucose concentration and the fluorescence level changes over time.

Conventional glucose sensors cannot accurately measure low glucose level in vivo. To test the microneedle sensor under lower glucose conditions, we induced hypoglycemia by insulin administration to fasted animals. The nano-diamond based device again demonstrated excellent correlation of the fluorescence signal with glucose level changes as illustrated in FIG. 10B.

The in vivo blood glucose level monitoring with the fluorescent nano-diamond based device was performed using 9 male mice (C57BL/6J, the Jackson Laboratory, 9-week-old), weighting 21˜16 g. All mice used in this study were bred and maintained at the ARC (animal resource center) of the University of Chicago in accordance with institutional guidelines. All the experimental procedures on live animals (mouse) were carried out in line with the Institutional Animal Care and Use Committee (IACUC) approved protocols of the Animal Care Center at the University of Chicago.

All the mice were housed under pathogen-free conditions in the ARC (Animal Resources Center) at the University of Chicago under a 12 h light-dark cycle. The housing facility maintained a temperature at 70-73° F. (average 72) and humidity at 40-50% (average 44%).

All the subjects were not involved in any previous procedures. Microneedle was sterilized prior to surgery using a 20 μl drop of 70% ethanol.

Before test, mice were anesthetized using intraperitoneal injection with 10 μl/g body weight drug cocktail (1 ml of Ketamine HCL (100 mg/ml, Hospira), 0.8 ml of xylazine (20 mg/ml, Akorn), and 8.2 ml of sterile water) and had their test site on dorsal shaved and sterilized with 70% ethanol.

We performed tests on four mice with 1˜3 glucose challenges or insulin challenges individually. For glucose challenge, mice were fasted for 8˜10 hours before the experiment. The emission light intensity from our device was recorded 5 min after the microneedle was inserted into the dorsal skin of mouse. Then, we injected 10 wt % glucose solution (1.5 g/kg glucose/body weight) into mice through intraperitoneal injection. 5 min after that, we measured blood glucose concentrations using a standard glucose meter (EZ meter; Bayer Contour NEXT) by blood sample from the snipped tail every 5 min. The emission light intensity from our device was also recorded every 5 min. For insulin challenge, mice were fasted for 8˜10 hours before the experiment. Then the mice were injected with 10 wt % glucose solution (1.5 g/kg glucose/body weight) through intraperitoneal injection. After 20 min, the microneedle was inserted into the dorsal skin of mouse. The emission light intensity from our device was recorded 5 min after. Then, we injected recombinant human insulin (2U/kg glucose/body weight) into mouse through intraperitoneal injection. 5 min after that, we measured blood glucose concentrations using a standard glucose meter by blood sample from the snipped tail every 5 min. The emission light intensity from our device was also recorded every 5 min.

All signals of the fluorescence intensity were averaged five times for each time point. We estimated the relative fluorescence intensity ΔFᵥᵢᵥₒ according to the below equation.

ΔFᵥᵢᵥₒ=100%×F÷F₀
F: Fluorescence intensity of the tested mouse
F₀: First fluorescence intensity of the tested mouse before glucose or insulin injection

Comparison between quantitative data were conducted using the unpaired or paired Student's t-test, Mann-Whitney U-test, or Dunnett's t-test, where appropriate. All P values were two-tailed and P values of 0.05 or less were considered to be statistically significant (*p<0.05, ** p<0.01, *** p<0.001).

Fluorescence intensity detected by the microneedle device continuously traced blood glucose level changes in glucose-challenged mice (as illustrated in FIG. 10A) or insulin-challenged mice (as illustrated in FIG. 10B) demonstrates that a microneedle device with a functionalized nano-diamond hydrogel network can be used to accurately monitor glucose changes in vivo in small animals. In FIGS. 10A-10D, glucose concentration is indicated by square data point symbols, and relative fluorescence intensity is indicated by diamond data point symbols.

The microneedle device exhibited similar response to glucose level changes when mounted on mouse skin during IPGTT and under hypoglycemia condition. As our device detects glucose concentration in skin interstitial fluid, there is a lag time of ˜ 5 minutes between the blood glucose level change and detection with our microneedle device as illustrated in FIGS. 10A and 10B.

To determine the stability of the device containing microneedles, a long-term photo-stability test was performed. The fluorescent nano-diamond based boronic hydrogel in the porous microneedles was prepared as described in Example 3. Then, it was kept in a clear microcentrifuge tube (Eppendorf, 1.5 ml, made of polypropylene) filled with PBS buffer under ambient light for 3 months. After that, the device was assembled and the glucose challenges or insulin challenges on four mice were tested again using the same method as described above. The results are illustrated in FIG. 10C (for glucose challenged mice) and in FIG. 10D (for insulin-challenged mice). Storage of the device for 3 months does not result in significant change in the time lag as well. Consistent with our observation in vitro, exposure to ambient light for up to three months does not affect the sensitivity of our sensor in vivo.

### Example 7: In Vivo Pig Study of Blood Glucose Level Monitoring

The in vivo blood glucose level monitoring with the fluorescent nano-diamond based device having porous microneedles with transparent barrels filled with fluorescent nano-diamond based boronic hydrogel prepared in accordance with Example 3 was performed using two male hybrid pigs, weighing 21 kg and 23 kg, respectively. All pig experimental procedures were approved by the Ethics Committee of Changhai Hospital, Shanghai, China. All pigs were housed under the temperature 24±2° C. and relative humidity 30-70% and given free access to food and water with a 12 h light-dark cycle. All the subjects were not involved in any previous procedures. Microneedle was sterilized prior to surgery using a 20 μl drop of 70% ethanol.

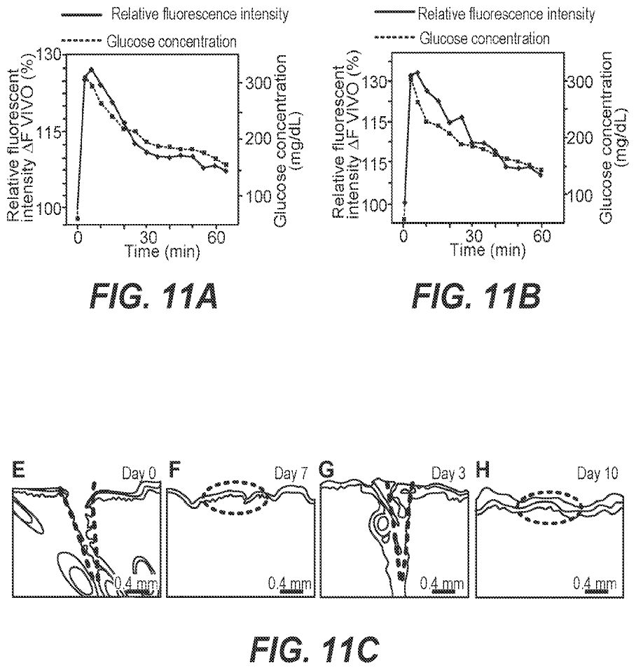

The pigs were anesthetized with an injection of ketamine (20 mg/kg, IM) and maintained with propofol (4 mg/kg/h). For stress-free and frequent blood sampling, central venous catheters were inserted into the external jugular vein. Pigs were fasted for 15 hours before the experiment. The emission light intensity from our device was recorded 5 min after the microneedle was inserted into the skin of front leg. After that, a bolus injection of 50% glucose solution was administered at 0.5 g/kg of body weight. The emission light intensity from our device was recorded at 0, 3, 6, 10, 15, 20, 25, 30, 35, 40, 45, 50, 55, 60, and 65 min after the microneedle was inserted into the dorsal skin of the pig. Results are graphically illustrated in FIG. 11A. Blood was collected at the indicated time points and blood glucose levels were measured with Cofoe glucometer (Hunan, China). In FIGS. 11A-11B, glucose concentration is indicated by square data point symbols, and relative fluorescence intensity is indicated by diamond data point symbols.

For long term in vivo blood glucose level monitoring test, the microneedle was left inserted in the skin of front leg for 72 hours. After that, the experiment was performed as above described. The emission light intensity from our device was recorded at 0, 3, 6, 10, 15, 20, 25, 30, 35, 40, 45, 50, 55, and 60 min after the injection of 50% glucose solution. Results are graphically illustrated in FIG. 11B. Blood was also collected at the indicated time points and blood glucose levels were measured with Cofoe glucometer (Hunan, China). Thus, the device demonstrated remarkable long-term photo and signal stability in vivo with large animals.

### Example 8: In Vivo Pig Study of Effects on Skin Pierced by Microneedle

Since porcine skin resembles human skin anatomically and physiologically, studies were performed to determine the effect of the microneedle device on porcine skin. It was confirmed that the microneedle device is minimally invasive to porcine skin. Only mild and localized erythema was observed immediately after needle application, which dissolved within minutes.

After the experiments, the experimented pig skin is cut off. The targeted area is the incised skin. Skin tissues were embedded in the Tissue-Tek optimal cutting temperature compound and cryosectioned into 5-μm slices. The tissue slabs were processed by standard histological procedures, histochemically-stained with hematoxylin and eosin (H&E), F4/80 antibody, trichrome, and CD³⁺ antibody. Antibodies were diluted according to manufacturer's instruction, unless indicated otherwise. Microscopic evaluation of the tissue sections was performed after that.

Histological examination of porcine skin revealed defined lesions in the epidermis and dermis immediately after needle insertion (FIG. 11C(E)), and complete wound healing seven days after removal (FIG. 11C(F)). Three days of continuous glucose monitoring with the microneedle device on free moving pigs showed no significant skin inflammation (FIG. 11C(G)) and the skin also totally recovered seven days after removing the device (FIG. 11C(H)).

### Example 9A: Fabrication of Fluorescent Nano-Diamond Hydrogel

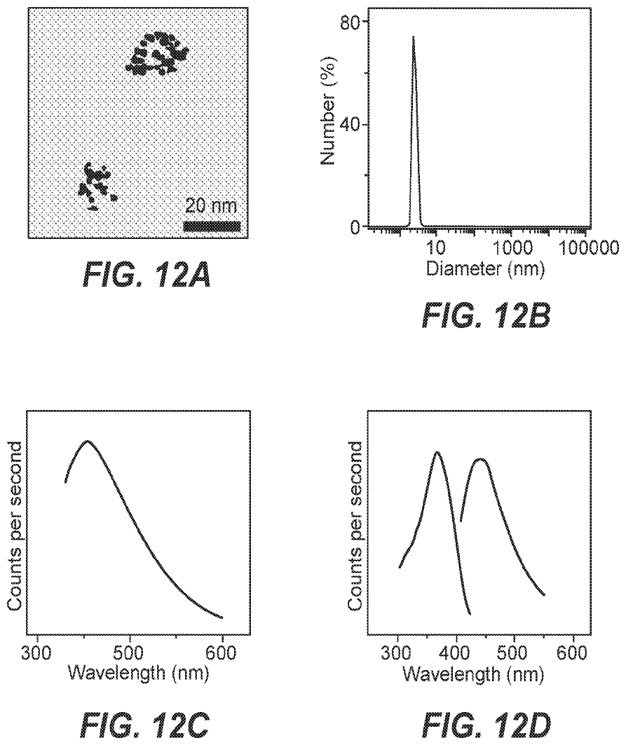

To prepare the octadecylamine modified nano-diamond, we used thionyl chloride to activate carboxyl group on the surface of the nano-diamond to generate acyl chloride and then reacted with amines to produce the amide. We further modified the nano-diamond using triethoxyvinylsilane to achieve alkene surface group for the covalent integration to the microneedle. The sequence of reaction was monitored by Fourier transform infrared (FTIR) spectroscopy to demonstrate unmodified nano-diamond, octadecylamine modified nano-diamond, and octadecylamine modified nano-diamond with alkene surface group. Weak peaks of the unmodified nano-diamond were noted at 2912 cm⁻¹ and the adjacent peak in the pristine nano-diamond can be attributed to the asymmetrical and symmetrical stretching vibration of hydrocarbon groups. The attachment of octadecylamine was verified by the dramatic increase of the intensity of hydrocarbon groups in modified nano-diamond, including the asymmetrical (2916 cm⁻¹) and symmetrical (2850 cm⁻¹) stretching vibrations, and the C—H scissor bending vibrations for methyl and methylene (1463 cm⁻¹; 1373 cm⁻¹). The amide bands at 1562 cm-1 and 1640 cm⁻¹ suggest covalent amide bond formation between octadecylamine and the nano-diamond. The strong Si—O—C stretching vibration bands at 1110 cm⁻¹ and 1020 cm¹ accompanied by a shoulder at 930 cm⁻¹ are indicative for the grafting of triethoxyvinylsilane on modified nano-diamond. The functionalized nano-diamond particles have an average size of 2±1 nm, as determined by transmission electron microscopy image (FIG. 12A) and dynamic light scattering (FIG. 12B). Octadecylamine modified nano-diamond has a maximum emission at 450 nm and maximum excitation at 370 nm in saline (FIGS. 12C and 12D).

To integrate nano-diamond into the boric hydrogel, we prepared a semi-transparent hydrogel by radical copolymerization of acrylamide, 3-(acrylamido)-phenylboronic acid, poly-(ethylene glycol) diacrylate, and triethoxyvinylsilane surface modified fluorescent nano-diamond on a glass bottom of tissue culture dish. In the absence of glucose, the neutral phenylboronic acid groups are hydrophobic and tend to interact well between each other, resulting in a high optical density of micro-heterogeneric polymer network. In the presence of glucose, glucose molecules can reversibly form 1:1 complex with the phenylboronic acid derivatives. The Donnan osmotic pressure of the hydrogel will increase. In the meantime, the density, hydration status, and refractive index of hydrogel change, leading to enhanced light propagation efficiency through the hydrogel. Consistent with this notion, confocal microscopy demonstrated differentially scattered nano-diamond particles in the hydrogel network at control (no glucose) or hyperglycaemic conditions (500 mg/dl of glucose) in vitro, demonstrating the rearrangement of hydrogel structure upon glucose concentration changes. As expected, this rearrangement of the boronic polymer network leads to enhanced light transmission and fluorescent emission under hyperglycaemic condition, as observed with a fluorescence microscope.

Quantitative analysis shows a nearly linear correlation between fluorescent emission of nano-diamond and glucose concentration in vitro. To examine the photostability of functionalized nano-diamond, we tested photobleaching in vitro in comparison with a classic glucose sensing hydrogel based on fluorescein isothiocyanate (FITC)-dextran and Concanavalin A (Con A). We found that exposure to excitation light (15 min, 5 W/cm², 460 nm) can lead to 6-10% bleaching of FITC-dextran fluorescence in the hydrogel, whereas no appreciable photobleaching was observed for nano-diamond or nano-diamond based boronic hydrogel in vitro. To assess the biocompatibility, we examined the potential cytotoxicity of our nano-diamond hydrogel. Our results show no significant changes of cell viability upon exposure to the hydrogel. Taken together, our results strongly suggest that the nano-diamond and boronic hydrogel material can serve as a safe and reliable biosensor for long term continuous glucose monitoring (CGM).

### Example 9B: Fabrication of Microneedles

The development of a miniaturized skin-wearable device with microneedle loading of nano-diamond hydrogel for CGM is desirable. However, the design and fabrication of a small but reliable device for optical detection of glucose are technically challenging.

To this end, we used porogen leaching technology to fabricate the microneedles with a porous hollow structure. The nano-diamond based boronic hydrogel was then covalently constructed in the bore of the microneedle after silanization of the microneedle bore surface. The wall of the constructed microneedle exhibits a random open pore structure up to ˜3 mm from the sharp tip. The blunt end of the microneedle (˜1 mm) was constructed without porogen, leading to an intact, uniform and transparent wall for an unfluctuating stable light transmission. The microneedle has a tip diameter of ˜180 μm, a base of ˜500 μm, and a length of ˜3 mm. The wall thickness is ˜20 μm at the tip and ˜120 μm at the base. The fabricated microneedles have a tip angle of ˜40°. The pore size of the microneedle ranges from 5 μm to 30 μm. The porous microneedles have randomly distributed but interconnected pores. The porous structure can effectively enhance extraction of interstitial fluid from the epidermis and dermis by capillary action, reducing lag time and facilitating glucose monitoring in vivo. The porous microneedle design may also reduce any pain which might be associated with device application, as it is minimally invasive.

Fluorescence imaging of the whole microneedle or cross-section of the needle showed that the microneedle itself does not have significant auto-fluorescence, whereas embedded nano-diamond hydrogel exhibited strong fluorescence signals.

### Example 9C: Fabrication of Microneedle Device

A miniature and skin-mountable device with the ability to transmit fluorescent signal from a minimally invasive microneedle has not been developed. To achieve these requirements, we used 3D printing approach to develop a light-weight, rugged, and skin mountable device for glucose monitoring. Specifically, the device includes: (1) a rugged, miniature part to conjugate two optical fibers with a microneedle for excitation and transmission of the fluorescent signal from the nano-diamond hydrogel embedded inside the microneedles, and (2) a portable optic assembly consisting of a light-emitting diode as an incident light source and an optical sensor chip as a detecting module. We tested the capability of glucose monitoring using our device assembled as described above in vitro. The fluorescence intensity at 450 nm was record with varying glucose concentrations (0-500 mg/dl) to verify the monitoring capability within the normal (80˜140 mg/dl), hypoglycemic (<80 mg/ml), and hyperglycemic (>140 mg/dl) ranges.^{25,26} When the glucose concentration increased from 0 mg/dl to 500 mg/dl, the fluorescence intensity collected by the device also increased. The nano-diamond hydrogel responds to the glucose concentration changes in a reversible manner. The microneedle device maintains its sensitivity to glucose when it is subjected to repeated exposure to glucose-free solution or glucose-containing solution (200 mg/dl). To determine the long-term photostability of our system, we kept the loaded microneedles in PBS buffer. Exposure to ambient light for up to 3 months does not compromise the capability of the microneedle device to sense the changes of glucose.

### REFERENCES

1. Wild S et al., “Global prevalence of diabetes: estimates for the year 2000 and projections for 2030,” Diabetes Care 2004; 27(5): 1047-1053.
2. Cefalu W T et al., “Update and next steps for real-world translation of interventions for type 2 diabetes prevention: reflections from a Diabetes Care editors' expert forum,” Diabetes Care 2016; 39: 1186-1201.
3. Ginter E & Simko V, “Type 2 diabetes mellitus, pandemic in 21st century,” in: Ahmad S. I. (eds), Diabetes: An Old Disease, a New Insight, series title: Advances in Experimental Medicine and Biology, 2013, vol. 771, pages 42-50, Springer (New York, NY).
4. Brownlee M, “Biochemistry and molecular cell biology of diabetic complications,” Nature 2001; 414(6865): 813-820.
5. Lee H et al., “A graphene-based electrochemical device with thermoresponsive microneedles for diabetes monitoring and therapy,” Nature Nanotechnology 2016; 11: 566-572.
6. Kondepati V R & Heise H M, “Recent progress in analytical instrumentation for glycemic control in diabetic and critically ill patients,” Analytical and Bioanalytical Chemistry 2007; 388(3): 545-563.
7. Klonoff D C, “Continuous glucose monitoring: roadmap for 21st century diabetes therapy,” Diabetes Care 2005; 28(5): 1231-1239.
8. Yue J et al., “Engineered epidermal progenitor cells can correct diet-induced obesity and diabetes,” Cell Stem Cell 2017; 21(2): 256-263.
9. Billingsley K et al., “Fluorescent nano-optodes for glucose detection,” Analytical Chemistry 2010; 82(9): 3707-3713.
10. Reineck P et al., “Effect of surface chemistry on the fluorescence of detonation nanodiamonds,” ACS Nano 2017; 11(11): 10924-10934.
11. Schrand A M et al., “Are diamond particles cytotoxic?,” The Journal of Physical Chemistry B 2007; 111(1): 2-7.
12. Yu S J et al., “Bright fluorescent nanodiamonds: no photobleaching and low cytotoxicity,” Journal of the American Chemical Society 2005; 127(50): 17604-17605.
13. Vaijayanthimala V et al., “The long-term stability and biocompatibility of fluorescent nanodiamond as an in vivo contrast agent,” Biomaterials 2012; 33(31): 7794-7802.
14. Mochalin V N & Gogotsi Y, “Wet chemistry route to hydrophobic blue fluorescent nanodiamond,” Journal of the American Chemical Society 2009; 131(3): 4594-4595.
15. Kuzimenkova M V et al., “Optical responses, permeability and diol-specific reactivity of thin polyacrylamide gels containing immobilized phenylboronic acid,” Polymer 2008; 49(6): 1444-1454.
16. Yetisen A K et al., “Glucose-sensitive hydrogel optical fibers functionalized with phenylboronic acid,” Advanced Materials 2017; 29(15): 1606380 (11 pages).

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

1. A device comprising:
a microneedle comprising a wall and an internal bore surrounded by the wall; and
a hydrogel disposed within the internal bore, wherein the hydrogel comprises a plurality of optically active particles and a plurality of capture agents.

2. The device of claim 1, wherein the wall comprises an optically transparent material.

3. The device of claim 1, wherein the wall comprises a porous material.

4. The device of claim 1, wherein the wall comprises glass, sapphire, diamond, ruby, silica, polycarbonate, poly(dimethylsiloxane), poly(vinyl chloride), poly(methyl methacrylate), polyethylene, and combinations thereof.

5. The device of claim 1, wherein the wall comprises an inner surface facing the internal bore, and wherein the inner surface is covalently bonded to the hydrogel.

6. The device of claim 1, wherein the hydrogel comprises poly(acrylamide) (PAAm), poly(acrylic acid) (PAA), poly(ethylene oxide) (PEO), poly(ethylene oxide)-block-poly(acrylic acid) (PEO-b-PAA), poly(N-isopropylacrylamide) (PNIPAAm), poly(ethylene oxide)-block-poly(N-isopropylacrylamide) (PEO-b-pNIPAAm), poly[poly(ethylene glycol) diacrylate] (p[PEGDA]), poly(acrylamide-co-poly(ethylene glycol) diacrylate) (p[AAM-co-PEGDA]), poly(lactic acid) (PLA), poly(lactic-co-glycolic acid) (PLGA), poly(caprolactone) (PCL), poly(aniline) (PANI), poly(N-(3-amidino)-aniline), poly(octamethylene citric acid), alginate, a poloxamer, poly(dimethylsiloxane), poly(butadiene), poly(isoprene), or a copolymer thereof.

7. (canceled)

8. The device of claim 1, wherein the plurality of optically active particles comprises a carbon-based material.

9. The device of claim 8, wherein the carbon-based material comprises nanodiamonds, carbon nanotubes, carbon nanowires, or carbon particles.

10. The device of claim 1, wherein the plurality of capture agents is configured to bind to an analyte or detect a condition selected from the group consisting of an ion, a small molecule, a metal, a metal ion, a metal atom, a particle, a metal particle, a magnetic particle, a temperature, a peptide, a protein, a cytokine, a hydrophilic sample, or a hydrophobic sample.

11. The device of claim 10, wherein the analyte is glucose, lactate, uric acid, glutathione, carbon dioxide, or hydrogen peroxide.

12. The device of claim 1, wherein the plurality of capture agents is selected from the group consisting of boronic acids, Schiff bases, acrylic acids, amides, amines, thiols, ionizable groups, reducible groups, charged groups, chelating groups, particles, magnetic particles, temperature responsive groups, redox indicators, photosensitizers, dyes, antibodies, nanostructures, and hydrophobic groups.

13. The device of claim 1, further comprising:
an optical source configured to transmit an optical input signal to the microneedle;
an optical sensor configured to receive an optical output signal from the microneedle; and
a controller comprising a memory and a processor, wherein the controller is configured to be electrically connected to the optical source and the optical sensor.

14. (canceled)

15. The device of claim 13, further comprising:
a wireless signal transmitter configured to transmit data from the controller or the memory to an external receiver.

16. The device of claim 1, further comprising a plurality of microneedles, wherein each of the plurality of microneedles comprises an internal bore and a hydrogel disposed therein.

17. The device of claim 1, wherein the microneedle is configured to obtain a sample comprising interstitial fluid from a subject.

18. The device of claim 1, wherein the microneedle extends from a planar substrate.

19. The device of claim 18, wherein the planar substrate comprises a flexible substrate.

20. The device of claim 19, wherein the flexible substrate comprises poly(dimethylsiloxane), poly(caprolactone), poly(lactic acid), or natural rubber.

21. A monitoring system comprising:
(i) a sampling component, which comprises:
a microneedle comprising a wall and an internal bore surrounded by the wall; and
a hydrogel disposed within the internal bore, wherein the hydrogel comprises a plurality of optically active particles and a plurality of capture agents; and
(ii) a detecting component, which comprises:
an optical source configured to transmit an optical input signal to the microneedle;
an optical sensor configured to receive an optical output signal from the microneedle; and
a controller comprising a memory and a processor, wherein the controller is configured to be electrically connected to the optical source and the optical sensor.

22. The monitoring system of claim 21, further comprising:
a first optical fiber configured to provide optical communication between the optical source and the microneedle, wherein the first optical fiber is configured to transmit the optical input signal; and
a second optical fiber configured to provide optical communication between the microneedle and the optical sensor, wherein the second optical fiber is configured to transmit the optical output signal.

23. The monitoring system of claim 21, further comprising:
(iii) a communicating component, which comprises:
a wireless signal transmitter configured to transmit data from the controller or the memory to an external receiver.

24. A method of detecting an analyte, the method comprising:
applying a microneedle to a target site of a subject, wherein the microneedle comprises a wall and an internal bore surrounded by the wall and further comprises a hydrogel disposed within the internal bore, and wherein the hydrogel comprises a plurality of optically active particles and a plurality of capture agents; and
measuring one or more optical output signals transmitted from the microneedle, wherein the one or more optical output signals are indicative of a presence or an absence of the analyte captured within the hydrogel.

25. The method of claim 24, wherein the target site is a dermal surface of the subject.

26. The method of claim 24, wherein said applying comprises providing access to interstitial fluid at the target site of the subject.

27. The method of claim 24, wherein said applying comprises affixing the microneedle at the target site for a period of about one to six months.

28. The method of claim 27, wherein the microneedle comprises an anti-inflammatory coating, an anti-immunogenic coating, or a biocompatible coating.

29. (canceled)

30. (canceled)

31. The method of claim 24 further comprising:
analyzing the one or more optical output signals to determine the presence or the absence of the analyte, thereby providing processed data; and
transmitting the processed data to an external receiver.
